(19) United States
(12) Patent Application Publication
(10) Pub. No.: US 2004/0259130 A1
(43) Pub. Date: Dec. 23, 2004

(54) Transgenesis of early embyonic cells

(76) Inventors: Alex J. Harvey, Athens, GA (US); Robert D. Ivarie, Watkinsville, GA (US)

Correspondence Address: AVIGENICS, INC., 111 RIVERBEND ROAD, ATHENS, GA, 30605, US

(21) Appl. No.: 10/812,248

(22) Filed: Mar. 29, 2004

Related U.S. Application Data
(60) Provisional application No. 60/458,699, filed on Mar. 28, 2003.

(51) Int. Cl. C12Q 1/68; C12N 15/85

(52) U.S. Cl. 435/6; 435/455

(57) ABSTRACT

The present invention includes methods of producing an integrated transgene in an avian cell which include introducing a nucleic acid into an avian cell by electroporating and methods of producing a transgenic avian comprising injecting the cell comprising the transgene into an avian stage X embryo. The present invention also provides for methods of screening for nucleic acid integration in a cellular genome which include transforming a nucleic acid comprising a marker into a recipient avian cell and determining if the nucleic is present in equal copy number in cells of a colony produced by the recipient avian cell.

### RELATED APPLICATIONS

The present application claims priority from U.S. provisional patent application Ser. No. 60/458,699, filed Mar. 28, 2003, which is hereby incorporated by reference in its entirety.

### FIELD OF THE INVENTION

The present invention relates to stem cells in general, and particularly relates to avian embryonic stem cells.

### BACKGROUND

The production of transgenic animals by introduction of exogenous genes into their germline has been achieved for a number of different animals including mice, cattle, rabbits, pigs, sheep, and fish. The introduction of exogenous genes into an animal's genome allows for the modification of the phenotypic characteristics of the animal. For example, the introduction of an appropriate transgene can potentially increase the disease resistance, growth rate, muscle mass or the like of an animal.

The production of transgenic birds is likewise highly desirable. Altering the avian genome can lead to the generation of desirable phenotypes. Furthermore, appropriate modification of the avian genome can lead to the production of exogenous protein within the oviduct followed by deposition of the exogenous proteins in the eggs of the bird. Use of the chicken as a bioreactor for the production of therapeutic proteins has significant advantages over the common methods of isolating proteins from natural sources and producing recombinant proteins in bacterial or mammalian cells.

Many attempts to introduce an exogenous expression construct into birds have involved the injection of retroviruses carrying non-viral transgenes into a freshly laid egg, just below the blastoderm (for examples, see Bosselman et al., Science, 1989, 243: 533-535; Salter et al., Virology, 1987, 157:236-240; Hughes et al., U.S. Pat. No. 4,997,763). Although, some success has been achieved using these procedures, complications can ensue. In some cases, efficient transduction of germline cells or expression of the inserted retroviral transgene has been problematic. In other cases, when the retroviral vector used is replication-competent, the genetically-modified chickens are viremic. Also, the size of the transgene in the retroviral vector is greatly limited.

In a different approach towards genetically altering birds, chimeric chickens have been generated by the injection of chicken blastodermal cells from one embryo into a recipient embryo, usually a stage X embryo. The donor blastodermal cells used in these experiments have been shown to be able to contribute to both somatic tissues (Watanabe et al., Development, 1992, 114:331-338; Fraser et al., Int. J. Devel. Biol., 1993, 37:381-385) and the germline (Thoraval et al., Poultry Sci., 1994, 73:1897-1905; Carsience et al., Development, 1993, 117:669-675; Petitte et al., Development, 1990, 108:185-189) of the resulting chimeras.

Although transgenic chickens can in theory be readily generated by the genetic manipulation of the donor embryonic cells prior to injection to the recipient blastoderm, the situation is greatly complicated by the fact that many sophisticated genetic manipulations require that the cell be maintained in culture over a period of time while the cells are screened for successful transfection, integration, or orientation of the transgene vector. In such experiments, it would be highly desirable to be able to culture the explant blastodermal cells for a sufficient amount of time to allow for the multiplication of embryonic stem cell progenitors without differentiation. However, it is very difficult to culture chicken embryonic or blastodermal cells for any period of time over approximately four days due to the tendency of chicken blastodermal cells to lose their ability to contribute to germline tissues of recipient embryos when cultured in vitro.

Examples of the difficulties of maintaining germline competence of chicken blastodermal cells in culture are detailed in Etches et al., Mol Reprod Dev, 1996, 45:291-8. In Etches et al., 1996, the frequency of contribution of cultured chicken blastodermal cells to germline and somatic chimeras upon injection to recipient embryos was compared to that of fresh chicken blastodermal cells. The chicken blastodermal cells were cultured under a variety of different conditions for 48 hours. Subjecting the chicken blastodermal cells to culturing for only 48 hours under any of the conditions tested resulted in a drop in germline contribution of over 60%.

Because of the problem of maintaining totipotent avian embryonic cells in culture, various attempts have been made by researchers to develop an effective method of determining the germline competence of the cells before addition to a recipient embryo. Several assays which attempt to identify totipotent chicken blastodermal cells have been reported (Karagenc et al., Dev. Genet., 1996, 19:290-301; Pain et al., Development, 1996, 122:2339-2348; Urven et al., Development, 1988, 103:299-304). These assays involve the detection of specific proteins thought to be characteristic of totipotent chicken blastodermal cells. Monoclonal antibodies specific to stage-specific embryonic antigen-1 (SSEA-1) or embryonal carcinoma Nulli SCC1 (EMA-1) are used in some of the assays (Karagenc et al., 1996; Urven et al., 1988). In Pain et al., 1996, the targeted protein is alkaline phosphatase. Such attempts are demonstrative of the need to sort germline-competent avian cells from those avian cells which have differentiated.

Similarly, due to the problems of maintaining avian embryonic cells in culture, attempts have also been made to alter the culture conditions to promote the stability of chicken embryonic cells in culture. For instance, U.S. Pat. No. 5,656,479, Petitte et al., teaches a procedure of growing avian stem cells on a mouse fibroblast feeder layer in the presence of a medium containing leukemia inhibitory factor (LIF) to generate a sustained avian stem cell culture. The success of such a procedure, however, is limited. For example, the Petitte et al. procedure was one of those tested in Etches et al., Mol. Reprod. Dev., 1996, 45:291-8, as described above.

Totipotent cells are necessary to generate germ-line transgenic avians, but genetically modifying cells in the short period they remain totipotent has proven difficult. For example, one problem has been determining which colonies have integrated the constructs into the genome in the short window of opportunity before the cells begin to differentiate.

Thus, there exists a need for a reliable methods of transforming avian cells. There also exists a need for screening transformed cells to determine whether the transformed nucleic acid is integrated into the recipient host cell genome.

### SUMMARY OF THE INVENTION

The present invention provides methods of transforming avian cells, for example, avian blastodermal cells, which may include transforming avian cells which possess the ability to give rise to germline tissue (e.g., totipotent cells). The invention also provides methods of screening avian cells for transformants having transformed nucleic acid integrated into their genome.

In one embodiment, the invention provides for methods of producing an integrated transgene in an avian cell, for example, a blastodermal cell, which includes introducing a nucleic acid into an avian cell by electroporation. The methods may include introducing a marker gene which is non-lethal. After electroporation, the cells may be allowed to undergo a cellular division. For example, the cells may be allowed to undergo 1 to about 1000 cellular divisions or 1 to about 100 cellular divisions or 3 to about 100 cellular divisions or 4 to about 100 cellular divisions or about 5 to about 100 cellular divisions or about 8 to about 100 cellular division or about 10 to about 100 cellular divisions or about 20 to about 100 cellular divisions or about 5 to about 20 cellular divisions. In one particularly useful embodiment, the transgene is stably integrated. The present invention also provides for methods to produce a transgenic avian by injecting the transformed avian cell into an avian embryo, for example, a stage X embryo. In one embodiment, the cell is injected into the embryo after passage (e.g., after cellular division). In one embodiment, the methods include allowing the cell to undergo a division in the presence of chick embryo extract.

The nucleic acid may include a marker gene. In one embodiment, the marker gene is a fluorescent expression marker (e.g., a fluorescent protein marker), for example, a GFP expression marker (e.g., an EGFP expression marker). In another embodiment, the marker is an antibiotic resistance gene, for example, a gene which encodes puromycin resistance.

In one embodiment, the electroporating introduces a double stranded break in a nucleic acid, for example, in the nucleic acid comprising the genome of an embryonic stem cell.

A coding sequence of the transgene may be expressed in any cell of the transgenic avian. For example, the coding sequence of the transgene may be expressed in the blood and/or sperm of the transgenic avian. In addition, a polypeptide encoded by a coding sequence of the transgene may be present in egg white produce by the transgenic avian. For example, the coding sequence may be for a light chain or a heavy chain of an antibody, (e.g., a human antibody). In one embodiment, the coding sequence is for a cytokine, for example, interferon.

The present invention provides for methods of screening transfected cells, for example, avian cells (e.g., avian blastodermal cells) for nucleic acid integration in a cellular genome. In one embodiment, an expression construct comprises the nucleic acid. These methods may include transfecting (e.g., transfecting by electroporation) a nucleic acid (e.g., DNA) comprising a marker into a recipient avian cell (e.g., a blastoderm) and determining if the nucleic acid is present in an equal copy number in cells of a colony produced by the recipient avian cell. For example, the nucleic acid may be present in an equal copy number in about 10% to 100% of the cells of a colony produced by a recipient avian cell or the nucleic acid may be present in an equal copy number in about 20% to 100% of the cells of a colony produced by a recipient avian cell or the nucleic acid may be present in an equal copy number in about 30% to 100% of the cells of a colony produced by a recipient avian cell or the nucleic acid may be present in an equal copy number in about 50% to 100% of the cells of a colony produced by a recipient avian cell or the nucleic acid may be present in an equal copy number in about 70% to 100% of the cells of a colony produced by a recipient avian cell or the nucleic acid may be present in an equal copy number in about 90% to 100% of the cells of a colony produced by a recipient avian cell or the nucleic acid may be present in an equal copy number in about 90% to 100% of the cells of a colony produced by a recipient avian cell or the nucleic acid may be present in an equal copy number in 100% of the cells of a colony produced by a recipient avian cell. In one particularly useful embodiment of the present invention, chick embryo extract is used in the cellular growth medium.

Certain methods of the invention allow for determining if the nucleic acid, which may include a transgene, is present in equal copy number in cells of a colony produced by the recipient avian cell include making such determination based on light emission. In one embodiment, the determination is made based on the use of a marker gene. Any marker gene that does not kill the cell is contemplated in the present invention. In one embodiment, the marker gene is a fluorescent expression marker (e.g., a fluorescent protein marker), for example, a GFP expression marker (e.g., an EGFP expression marker). In other embodiments, β-lactamase or β-galactosidase is used as the marker.

In one embodiment, determining if the nucleic is present in an equal copy number in cells of a colony produced by the recipient avian cell may be accomplished by determining if a marker is homogeneously present in cells of a colony produced by the recipient cell. In one embodiment, the marker is present homogeneously in cells of a colony produced by the recipient cell indicating the nucleic acid is integrated in the genome of the recipient host cell. In another embodiment, the marker is present non-homogeneously in cells of a colony produced by the recipient cell indicating the nucleic acid is not integrated in the genome of the recipient host cell.

Any combination of features described herein are included within the scope of the present invention provided that the features included in any such combination are not mutually inconsistent as will be apparent from the context, this specification, and the knowledge of one of ordinary skill in the art.

Additional objects and aspects of the present invention will become more apparent upon review of the detailed description set forth below when taken in conjunction with the accompanying figures, which are briefly described as follows.

### BRIEF DESCRIPTION OF THE DRAWINGS

FIG. 1. Map of vectors used in the transformation of embryonic chicken cells. A) Map of the essential components of the screening (enhanced green fluorescent protein, EGFP) or selection (puromycin resistance gene, pac) vector. B) Map of the ovalbumin targeting vector pOVTV-7.4/0.785-IFNMM-RSV-pur (EGFP). Sites of the promoter region are denoted to indicate the 5′ arm of the targeting vector. The 3′ end of the targeting vector overlaps exon L and intron 1.

FIG. 2. Nonhomogenous and homogenous EGFP CBC colonies. A) UV light image of a CBC culture. Expression of the green fluorescent protein in the CBC colonies is variegated or nonhomogenous. Scale bar, 100 uM B) Visible image of the CBC colonies on a STO feeder shown in A. C) Homogenous CBC-EGFP positive colony. About 90% of the cells in the colony express green fluorescent protein. D) Visible image of C. E) Homogenous CBC-EGFP positive colony. >70% of the cells in the colony express green fluorescent protein. F) Visible image of E.

FIG. 3. EGFP positive CBCs harvested from 4 day-cultured homogenous green fluorescent CBC colonies prior to injection into stage X embryos. A) UV image of harvested cells. Scale bar, 10 uM. B) Visible image of A. CBCs were stained by trypan blue; the small cells are CBCs; the bigger cells are STOs and blue cells are dead cells.

FIG. 4. CBCs-EGFP colonies: A) BDCs were electroporated with pOVTV-7.4/0.785-IFNMM-RSV-EGFP, vector linearized with Sac II, cultured for four days, homogenous green fluorescent colonies picked and passaged once. Above pictures show that two among four colonies retained EGFP. These colonies were from homogenous EGFP+ colonies. The EGFP negative colonies were from contaminating colonies that had a variegated EGFP expression pattern. Scale bar, 100 uM. B) visible image of A. C) Passage 4 of CBC EGFP+ colonies. CBC-EGFP colonies at passage 2 were picked and passaged. All CBC colonies are EGFP positive. D) Visible image of C. E).Passage 6 of CBC EGFP+ colonies. All CBC colonies are EGFP positive. F) Visible image of E.

FIG. 5. Puromycin selection of WEFs A) WEFs prior to addition of puromycin. Scale bar, 400 uM. B) A typical WEF colony 7 days after addition of puromycin. Scale bar, 100 uM.

FIG. 6. There are primarily 6 different types of CBC colonies after 8 to 9 days of puromycin selection. Scale bar, 100 uM.

FIG. 7. A) CBCs colonies in passage 2 from a single Puromycin resistant type 2 CBC colony, six days. Scale bar, 100 uM. B) CBCs colonies are at passage 3 from a single puromycin resistant type 2 colony, five days. C) CBCs colonies are at passage 4 from a single puromycin resistant type 2 colony, six days.

FIG. 8. pac composites carry the transgene in their blood DNA. Extracted blood DNA from composites and non-transgenic chicks were analyzed by real-time PCR using the neomycin primer/probe set.

FIG. 9. Anatomy of a single copy random insertion. Top line is representative of the pOVTV-7.4/0.785-IFNMM-RSV-pur (EGFP) transgene, denoted by the bracket, integrated into a region of a chicken chromosome. The transgene has a single BamH I site between the IFN and pac coding sequences. In this example, the transgene integrated between two BamH I sites that were, prior to integration, 13 kb apart. In a Southern blot analysis of this example, a IFN probe should detect a BamH I-digested fragment of 17.5 kb and a pac probe should detect a fragment of 9.5 kb.

FIG. 10. Transgene Integration in pur-resistance and EGFP+ CBCs. DNA was extracted from passaged CBCs, digested by BamH I, separated by agarose gel electrophoresis, transferred to a membrane and probed with the IFN coding sequence. CTRL is from non-transgenic CBCs. Lanes 1-8 are from puromycin resistant CBC colonies. Lane 9 is from an EGFP positive colony at passage eleven. Lane 10 is from non-transgenic whole embryo fibroblasts. White arrows mark denote bands that correspond to the transgene. The dark band at ˜12 kb and the lighter bands that are present in all samples are genomic DNAs to which the probe non-specifically hybridized. The arrow at 8.5 kb indicates the minimum size of a transgene that is intact and integrated into the chicken genome.

FIG. 11. Transgene Integration in pur-resistance and EGFP+ CBCs. A) DNA was extracted from passaged CBCs, digested by BamH I, separated by agarose gel electrophoresis, transferred to a membrane and probed with the IFN sequence, stripped and reprobed with the pac coding sequence. CTRL is from non-transgenic CBCs. Lanes 1-8 are from puromycin resistant CBC colonies. Lane 9 is from an EGFP positive colony at passage eleven. Lane 10 is from non-transgenic whole embryo fibroblasts. Black arrows denote bands that correspond to the transgene. The light band at ˜12 kb and the lighter bands that are present in all samples are genomic DNAs that non-specifically hybridized to the IFN probe and that did not completely wash during the stripping. The arrow at 5.5 kb indicates the minimum size of a transgene that is intact and integrated into the chicken genome. The dark band at ˜14.5 kb in lane 3 is likely the same band detected by the IFN probe and was not efficiently stripped. B) Enhanced image of the light bands in lanes 5 and 8.

FIG. 12. Comparison of junction fragments in pur-resistance and EGFP+ CBCs. The membrane probed with IFN is marked with the position of bands detected by the pac probe (black bars).

Definitions and Abbreviations

The following definitions and abbreviations are set forth to illustrate and define the meaning and scope of the various terms used to describe the invention herein.

A “BDC” is a blastodermal cell.

A “CBC” is a cultured blastodermal cell.

A “GFP” is a green fluorescent protein.

An “EGFP” is an enhanced green fluorescent protein.

An “ES” cell is an embryonic stem cell.

A “WEF” is a whole embryo fibroblast.

A “germline-competent” cell is a cell which can contribute to germline tissue. A germline-competent cell may, but need not necessarily be, a totipotent cell. Alternatively, a germline-competent cell can be a pluripotent cell.

A “totipotent” cell, as used herein, is a cell capable of giving rise to all types of differentiated cells found in the particular organism from which the cell originated. A totipotent cell is also a cell capable of contributing to germline tissue.

A “pluripotent” cell is a cell capable of differentiating into more than one different final differentiated types. A pluripotent cell may or may not be capable of contributing to germline tissue.

“Germline tissue”, as used herein, refers to cells of the reproductive organs from which sperm or oocytes are formed.

A “marker” or “marker gene” is a gene which encodes a protein that allows for identification and isolation of correctly transfected cells. Suitable marker sequences include, but are not limited to those encoding green, yellow, and blue fluorescent protein (the GFP, YFP, and BFP genes, respectively). Other suitable markers include genes encoding thymidine kinase (tk), dihydrofolate reductase (DHFR), and aminoglycoside phosphotransferase (APH). The latter imparts resistance to the aminoglycoside antibiotics, such as kanamycin, neomycin, and geneticin. Use of a neomycin resistance gene as a marker is particularly suitable. Other marker genes include those encoding chloramphenicol acetyltransferase (CAT), β-lactamase, and β-galactosidase (β-gal). A “reporter gene” is a marker gene that “reports” its activity in a cell by the presence of the protein that it encodes.

A gene which is “substantially expressed only in cells which are germline-competent” is a gene which shows at least an approximately 5-fold higher level of expression (as evidenced by protein levels) in cells which are germline-competent than in cells which are not germline-competent under a given set of conditions. Preferably, the gene shows at least an approximately 10-fold higher level of expression in cells which are germiline-competent than in cells which are not germline-competent.

“Operably or operatively linked” refers to the configuration of the coding and/or control sequences so as to perform the desired function. Thus, control sequences operably linked to a coding sequence are capable of effecting the expression of the coding sequence. A coding sequence is operably linked to or under the control of transcriptional regulatory regions in a cell when RNA polymerase will bind the control sequence and transcribe the coding sequence into mRNA that can be translated into the encoded protein. The control sequences need not be contiguous with the coding sequence, so long as they function to direct the expression thereof. Thus, for example, intervening untranslated yet transcribed sequences can be present between a control sequence and the coding sequence and the promoter sequence can still be considered “operably linked” to the coding sequence. Furthermore, when a control sequence (such as an enhancer) is said to be operably linked to another control sequence (such as a promoter), the sequences are capable of working together to regulate expression of the coding sequence.

The term “control sequences” refers herein to nucleic acid sequences which control transcription of a given gene. For instance, control sequences include those sequences required to initiate or terminate gene transcription plus those sequences which (positively or negatively) regulate the rate at which transcription initiation occurs. Examples of control sequences in eukaryotic cells include, but are not limited to, promoters, enhancers, and repressor binding sites.

“Vector” means a polynucleotide comprised of single strand, double strand, circular, or supercoiled DNA or RNA. A typical vector may be comprised of the following elements operatively linked at appropriate distances for allowing functional gene expression: replication origin, promoter, enhancer, 5′ mRNA leader sequence, ribosomal binding site, nucleic acid cassette, termination and polyadenylation sites, and selectable marker sequences. One or more of these elements may be omitted in specific applications. The nucleic acid cassette can include a restriction site for insertion of the nucleic acid sequence to be expressed. In a functional vector the nucleic acid cassette contains the nucleic acid sequence to be expressed including translation initiation and termination sites. An intron optionally may be included in the construct, preferably ≧100 bp 5′ to the coding sequence.

A “coding sequence” refers to a polynucleotide or nucleic acid sequence which can be transcribed and translated (in the case of DNA) or translated (in the case of mRNA) into a polypeptide in vitro or in vivo when placed under the control of appropriate control sequences. The boundaries of the coding sequence are determined by a translation start codon at the 5′ (amino) terminus and a translation stop codon at the 3′ (carboxy) terminus. A transcription termination sequence will usually be located 3′ to the coding sequence. A coding sequence may be flanked on the 5′ and/or 3′ ends by untranslated regions.

An “expression construct” or “expression vector” is a vector which is constructed so that the particular coding sequence is located in the vector with the appropriate control sequences including regulatory sequences, the positioning and orientation of the coding sequence with respect to the control sequences being such that the coding sequence is transcribed under the “control” of the control or regulatory sequences. Modification of the sequences encoding the particular protein of interest may be desirable to achieve this end. For example, in some cases it may be necessary to modify the sequence so that it may be attached to the control sequences with the appropriate orientation; or to maintain the reading frame. The control sequences and other regulatory sequences may be ligated to the coding sequence prior to insertion into a vector. Alternatively, the coding sequence can be cloned directly into an expression vector which already contains the control sequences and an appropriate restriction site which is in reading frame with and under regulatory control of the control sequences.

### DETAILED DESCRIPTION

The present invention provides for methods of producing transgenic avians. The methods include introducing a nucleic acid which may include a transgene of interest into avian cells, for example, avian embryonic cells (e.g., early avian embryonic cells) resulting in integration of the all or part of the nucleic acid into the genome of the cell. The methods may also include introducing the transformed avian embryonic cells into an avian embryo, for example, an avian stage X embryo, for the production of a transgenic chicken. The present invention also relates to transgenic avians, and to eggs laid by such transgenic avians, produced according to methods of the invention.

The present invention provides for the introduction of a nucleic acid into avian cells which includes a marker gene that allows for the non-lethal detecting or the selecting of a cell in which the nucleic acid, in part or all, has incorporated into the genome of the cell. However, the exact nature of the nucleic acid or transgene of interest introduced into the avian cells is not critical to the present invention. Many operably linked combinations of a promoter (optionally in combination with other control sequences) which is active in avian animals and a coding sequence may be employed in the present invention. The coding sequence of the transgene may encode an exogenous protein or peptide. Alternatively, the coding sequence may encode an antisense RNA molecule or a ribozyme. The promoter on the transgene may be constitutive, tissue-specific, or inducible. The expression vectors described in WO99/19472, herein incorporated by reference, can optionally be used as transgenes in the present invention.

In one embodiment, the present invention provides for transforming an avian cell, for example, an embryonic avian cell (e.g., early embryonic avian cells) by electroporating nucleic acids into the cell. The avian cell may be, for example, a cell of a stage I avian embryo, a cell of a stage II avian embryo, a cell of a stage III avian embryo, a cell of a stage IV avian embryo, a cell of a stage V avian embryo, a cell of a stage VI avian embryo, a cell of a stage VII avian embryo, a cell of a stage VIII avian embryo, a cell of a stage IX avian embryo, a cell of a stage X avian embryo, a cell of a stage XI avian embryo or a cell of a stage XII avian embryo. In one particularly useful embodiment, the avian cell is a cell of a stage X avian embryo.

Electroporation may provide for double-stranded breaks which facilitate integration of nucleic acid which may comprise a transgene into the genome. This is an advantage over lipofection transformation techniques often used in avian transgenesis.

In one embodiment, the invention provides for electroporation as the transfection technique and GFP (e.g., EGFP) based screening for cells that have taken up the nucleic acid or transgene of interest. Incorporation of the sequence coding for GFP allows screening of avian cells, such as cultured blastodermal cells, with the desired genetic modification based on green fluorescence.

In one embodiment, electroporation of 10⁷ blastodermal cells yields, after growth for four days, 20-30 colonies that express EGFP in all or most of the cells in a colony. Significantly, the intensity of EGFP expression is uniform, or homogeneous, cell to cell in the colonies, which suggests that each cell has the same copy number of the EGFP expression cassette. Colonies which have variegated EGFP expression may have unequal numbers of the transgene in each cell, which may be due to, for example, and without limitation, episomal propagation and unequal partitioning of the plasmid during cellular division.

CBC colonies that express EGFP in all or most of the colony's cells during or after the four-day culture window are easily produced according to the present methods. One advantage of this screening method instead of selection is that it doesn't kill any cells. Another advantage is that it works within four days. Electroporation in combination with EGFP may show at least a 100-fold increase in the number of EGFP-positive colonies after four days of culture compared to standard lipofection transformation techniques. Colonies in which all or most cells express EGFP indicate integration of the transgene opposed to episomal replication. Thus, the homogeneity of the fluorescence across a colony provides a visual indication of which colonies include cells with an integrated transgene (i.e., a transgene integrated into the genome of the cells).

In one embodiment, the invention provides for electroporation as the transfection technique in combination with antibiotic screening. For example, screening for cells that have taken up a transgene may be accomplished through use of puromycin. Incorporation of the sequence coding for puromycin resistance allows for the screening of transformed avian cell, for example, cultured blastodermal cells, in which the transformed DNA which comprises the puromycin resistance has integrated.

A number of drug resistance genes have been studied for their ability to rapidly select for BDCs that carried the desired plasmid post-transfection. A striking success was seen the puromycin-resistance gene or pac. Low concentrations of the puromycin (0.5 to 2.0 micrograms/milliliter) completely killed BDCs in 2 days. Two different vectors have been examined which express pac with two different promoters, both of which efficiently induced resistance in BDCs. The result is that selection of CBC colonies with 10 to 50 cells each forming in 2 days may be obtained.

It may be important in drug-based selection to kill non-resistant cells quickly (within a few days) through use of a fast-acting drug such as puromycin. Certain other antibiotics such as neomycin do not appear to be effective in this regard.

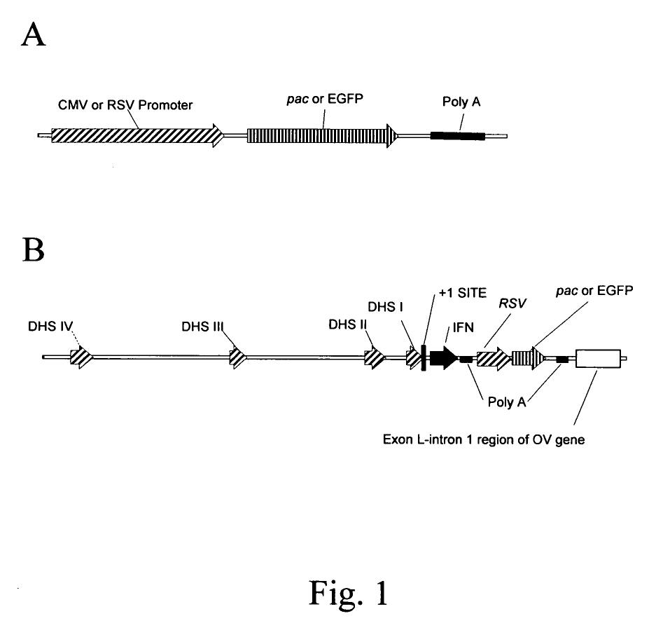

Transgenic blastodermal cells and chimeric chickens with the 7.4 kb ovalbumin promoter and human interferon gene have been produced by a puromycin selection method in combination with electroporation. To select for the transgenic cells a puromycin (pac) resistance gene was included in the construct (FIG. 1) which was introduced it into stage X blastodermal cells by electroporation. However, the present invention is not limited to use of any particular construct. That is, the puromycin resistance gene may be employed on any suitable construct for use in the present invention. In addition, the invention contemplates the use of all avian cells including without limitation, embryonic cells stage I to stage XII. By growing the cells in the presence of a low concentration of puromycin, cells that have not taken up the transgene were killed, for example, after six days. Only colonies of cells stably bearing the transgene survived.

An RSV promoter may work well for the pac gene. For example, RSV has the advantage of being smaller than promoters others have used (such as the beta-actin promoter fused to RSV enhancer). A smaller promoter makes more space available for a larger transgene.

Temperature is important to maintain an elevated division rate of the cultured blastodermal cells. An increased rate of division may be desirable because more cells are then available for the production of composites (chimeras). In addition, a faster division rate may facilitate removal of episomal plasmid DNA as any DNA that has not integrated into a cellular genome will not replicate efficiently and may be diluted away or degraded after each division. Faster removal of episomal plasmid DNA may provide shorter culture times, which in turn may increase the likelihood of germline transmission of the transgene.

Growing chicken cultured blastodermal cells at temperatures higher than 37° C. may result in more rapidly proliferating and generally healthier cells than those grown at lower temperatures. In particular, chicken cells grown at 39.5° C. (i.e., between 37° C. and 41° C., the physiological temperatures of chickens and mammals) divide 25-40% faster than those grown at 37° C. In addition, the murine-derived STO feeder cells survive these temperatures. Surprisingly, STOs are resistant to puromycin for a period of time, for example, 10 days. As an alternative source of feeder cells, chicken granulosa cells, which grow well at high temperatures, and fortuitously are also puromycin-resistant may be used.

Methods for transferring an avian embryonic cell, in some cases following genetic manipulations to an avian embryo, are well known to those skilled in the art. Typically, portions of the shell and outer shell membranes are removed from the recipient embryo's egg to expose the embryo. For example, an opening about 5 mm in diameter may be made in the side of an egg, normally by the use of a drilling tool fitted with an abrasive rotating tip which can drill a hole in the egg shell without damaging the underlying shell membrane. The membrane is then cut out by use of a scalpel. The genetically altered embryonic cells are then injected into the egg containing the embryo. The cell or cells may be injected into the yolk sac or onto the chorioallantoic membrane, preferably into the subgerminal cavity, and preferably during early embryonic development such as prior to day 2 or 3 of incubation, and most preferably prior to day 1 of incubation. Examples of methods for transferring avian cells to recipient embryos can be found in the following references, all of which are herein incorporated by reference: Watanabe et al., Development, 1992, 114:331-338; Fraser et al., Int. J. Devel. Biol., 1993, 37:381-385; Thoraval et al., Poultry Sci., 1994, 73:1897-1905; Carsience et al., Development, 1993, 117:669-675; Petitte et al., 1990, 108:185-189; U.S. Pat. No. 5,656,479; Brazolot et al., Molecular Reproduction and Development, 1991, 30:304-312; and U.S. Pat. No. 5,897,998.

The step of allowing the avian embryo to which transformed cell has been transferred to develop to hatch is routine for one of ordinary skill in the art. For instance, if the egg has been windowed, then the opening in the egg is typically resealed with shell membrane and a sealing material, preferably glue or paraffin. The sealed egg is then incubated first at 37.5° C. for a few days, and then at 37° C. until hatch.

Many possible applications of the methods of producing transgenic avian animal exist. For example, the present methods may be useful for producing genetically engineered avian (chickens, quail, turkey, duck etc.) cell lines for use in cloning or nuclear transfer or for the production of any genetically engineered avians through culture of embryonic chicken cells. In one embodiment, the present invention is used to create a line of germline-modified transgenic chickens which may express exogenous proteins in their oviducts and deposit those exogenous proteins in their eggs. In another embodiment, the present invention is used to create a line of chimeric transgenic chickens which may express exogenous proteins in their oviducts and deposit those exogenous proteins in their eggs.

The present invention also provides for methods of screening transformed cell populations for cells in which the transformed nucleic acid, in part or all, has integrated into the host cell's genome.

Another aspect of the present invention is a method of expressing a heterologous polypeptide in an avian cell by stably transforming a cell by, as described above, and culturing the transfected cell under conditions suitable for expression of the heterologous polypeptide under the control of the avian transcriptional regulatory region.

The protein of the present invention may be produced in purified form by any known conventional techniques. For example, chicken cells, an egg or an egg white may be homogenized and centrifuged. The supernatant may then be subjected to sequential ammonium sulfate precipitation and heat treatment. The fraction containing the protein of the present invention is subjected to gel filtration in an appropriately sized dextran or polyacrylamide column to separate the proteins. If necessary, the protein fraction may be further purified by HPLC or other methods well known in the art of protein purification.

The methods of the invention are useful for expressing nucleic acid sequences that are optimized for expression in avian cells and which encode desired polypeptides or derivatives and fragments thereof. Derivatives include, for instance, polypeptides with conservative amino acid replacements, that is, those within a family of amino acids that are related in their side chains (commonly known as acidic, basic, nonpolar, and uncharged polar amino acids). Phenylalanine, tryptophan, and tyrosine are sometimes classified jointly as aromatic amino acids and other groupings are known in the art (see, for example, “Biochemistry”, 2nd ed, L. Stryer, ed., W. H. Freeman & Co., 1981). Peptides in which more than one replacement has taken place can readily be tested for activity in the same manner as derivatives with a single replacement, using conventional polypeptide activity assays (e.g. for enzymatic or ligand binding activities).

Regarding codon optimization, if the recombinant nucleic acid molecules are transfected into a recipient chicken cell, the sequence of the nucleic acid insert to be expressed can be optimized for chicken codon usage. This may be determined from the codon usage of at least one, and preferably more than one, protein expressed in a chicken cell according to well known principles. For example, in the chicken the codon usage could be determined from the nucleic acid sequences encoding the proteins such as lysozyme, ovalbumin, ovomucin and ovotransferrin of chicken. Optimization of the sequence for codon usage can elevate the level of translation in avian eggs.

The present invention further relates to methods for gene expression by avian cells from nucleic acid vectors, and transgenes derived therefrom, that include more than one polypeptide-encoding region wherein, for example, a first polypeptide-encoding region can be operatively linked to an avian promoter and a second polypeptide-encoding region is operatively linked to an Internal Ribosome Entry Sequence (IRES). It is contemplated that the first polypeptide-encoding region, the IRES and the second polypeptide-encoding region of a recombinant DNA of the present invention may be arranged linearly, with the IRES operably positioned immediately 5′ of the second polypeptide-encoding region. This nucleic acid construct, when inserted into the genome of an avian cell or a bird and expressed therein, will generate individual polypeptides that may be post-translationally modified and combined in the white of a hard shell bird egg. Alternatively, the expressed polypeptides may be isolated from an avian egg and combined in vitro.

The invention, therefore, includes methods for producing multimeric proteins including immunoglobulins, such as antibodies, and antigen binding fragments thereof. Thus, in one embodiment of the present invention, the multimeric protein is an immunoglobulin, wherein the first and second heterologous polypeptides are immunoglobulin heavy and light chains respectively. Illustrative examples of this and other aspects of the present invention for the production of heterologous multimeric polypeptides in avian cells are fully disclosed in U.S. patent application Ser. No. 09/877,374, filed Jun. 8, 2001, by Rapp, published as US-2002-0108132-A1 on Aug. 8, 2002, and U.S. patent application Ser. No. 10/251,364, filed Sep. 18, 2002, by Rapp, both of which are incorporated herein by reference in their entirety.

Accordingly, the invention further provides immunoglobulin and other multimeric proteins that have been produced by transgenic avians of the invention.

In various embodiments, an immunoglobulin polypeptide encoded by the transcriptional unit of at least one expression vector may be an immunoglobulin heavy chain polypeptide comprising a variable region or a variant thereof, and may further comprise a D region, a J region, a C region, or a combination thereof. An immunoglobulin polypeptide encoded by an expression vector may also be an immunoglobulin light chain polypeptide comprising a variable region or a variant thereof, and may further comprise a J region and a C region. The present invention also contemplates multiple immunoglobulin regions that are derived from the same animal species, or a mixture of species including, but not only, human, mouse, rat, rabbit and chicken. In preferred embodiments, the antibodies are human or humanized.

In other embodiments, the immunoglobulin polypeptide encoded by at least one expression vector comprises an immunoglobulin heavy chain variable region, an immunoglobulin light chain variable region, and a linker peptide thereby forming a single-chain antibody capable of selectively binding an antigen.

Examples of therapeutic antibodies that may be produced in methods of the invention include but are not limited to HERCEPTIN™ (Trastuzumab) (Genentech, CA) which is a humanized anti-HER2 monoclonal antibody for the treatment of patients with metastatic breast cancer; REOPRO™ (abciximab) (Centocor) which is an anti-glycoprotein IIb/IIIa receptor on the platelets for the prevention of clot formation; ZENAPAX™ (daclizumab) (Roche Pharmaceuticals, Switzerland) which is an immunosuppressive, humanized anti-CD25 monoclonal antibody for the prevention of acute renal allograft rejection; PANOREX™ which is a murine anti-17-IA cell surface antigen IgG2a antibody (Glaxo Wellcome/Centocor); BEC2 which is a murine anti-idiotype (GD3 epitope) IgG antibody (ImClone System); IMC-C225 which is a chimeric anti-EGFR IgG antibody (ImClone System); VITAXIN™ which is a humanized anti-αVβ3 integrin antibody (Applied Molecular Evolution/MedImmune); Campath 1H/LDP-03 which is a humanized anti CD52 IgG1 antibody (Leukosite); Smart M 195 which is a humanized anti-CD33 IgG antibody (Protein Design Lab/Kanebo); RITUXAN™ which is a chimeric anti-CD2O IgG1 antibody (IDEC Pharm/Genentech, Roche/Zettyaku); LYMPHOCIDE™ which is a humanized anti-CD22 IgG antibody (Immunomedics); ICM3 is a humanized anti-ICAM3 antibody (ICOS Pharm); IDEC-114 is a primate anti-CD80 antibody (IDEC Pharm/Mitsubishi); ZEVALIN™ is a radiolabelled murine anti-CD20 antibody (IDEC/Schering AG); IDEC-131 is a humanized anti-CD40L antibody (IDEC/Eisai); IDEC-151 is a primatized anti-CD4 antibody (IDEC); IDEC-152 is a primatized anti-CD23 antibody (IDEC/Seikagaku); SMART anti-CD3 is a humanized anti-CD3 IgG (Protein Design Lab); 5G1.1 is a humanized anti-complement factor 5 (CS) antibody (Alexion Pharm); D2E7 is a humanized anti-TNF-α antibody (CATIBASF); CDP870 is a humanized anti-TNF-α Fab fragment (Celltech); IDEC-151 is a primatized anti-CD4 IgG1 antibody (IDEC Pharm/SmithKline Beecham); MDX-CD4 is a human anti-CD4 IgG antibody (Medarex/Eisai/Genmab); CDP571 is a humanized anti-TNF-α IgG4 antibody (Celltech); LDP-02 is a humanized anti-α4β7 antibody (LeukoSite/Genentech); OrthoClone OKT4A is a humanized anti-CD4 IgG antibody (Ortho Biotech); ANTOVA™ is a humanized anti-CD40L IgG antibody (Biogen); ANTEGREN™ is a humanized anti-VLA-4 IgG antibody (Elan); and CAT-152 is a human anti-TGF-β₂ antibody (Cambridge Ab Tech).

One aspect of the present invention, therefore, concerns transgenic birds, such as chickens, comprising a recombinant nucleic acid molecule and which preferably (though optionally) express a heterologous gene in one or more cells in the animal. Suitable methods for the generation of transgenic avians having heterologous DNA incorporated therein are described, for example, in WO 99/19472 to Ivarie et al.; WO 00/11151 to Ivarie et al.; and WO 00/56932 to Harvey et al., all of which are incorporated herein by reference in their entirety.

Embodiments of the methods for the production of a heterologous polypeptide by the avian tissue such as the oviduct and the production of eggs which contain heterologous protein involve providing a suitable vector and introducing the vector into embryonic blastodermal cells so that the vector can integrate into the avian genome, for example by electroporation. A subsequent step involves deriving a mature transgenic avian from the transgenic blastodermal cells produced in the previous steps. Deriving a mature transgenic avian from the blastodermal cells optionally involves transferring the transgenic blastodermal cells to an embryo and allowing that embryo to develop fully, so that the cells become incorporated into the bird as the embryo is allowed to develop. Another alternative is to transfer a transfected nucleus to an enucleated recipient cell which may then develop into a zygote and ultimately an adult bird. The resulting chick is then grown to maturity.

It is contemplated, for example, that the recombinant nucleic acid molecules of the present invention may be introduced into a blastodermal embryo by electroporation of the DNA into a stage X or earlier embryo cell that has been removed from the oviduct. The cell is returned to an avian embryo which is then returned to the bird for egg white deposition, shell development and laying. The resulting embryo is allowed to develop and hatch, and the chick allowed to mature. In one embodiment, a chimeric transgenic chick is produced. In another embodiment, a germ line transgenic chick is produced.

A transgenic bird produced from the transgenic blastodermal cells may be known as a “founder.” Some founders can be chimeric or mosaic birds if, for example, transformation does not deliver nucleic acid molecules to all of the blastodermal cells of an embryo. Some founders will carry the transgene in the tubular gland cells in the magnum of their oviducts and will express the heterologous protein encoded by the transgene in their oviducts. If the heterologous protein contains the appropriate signal sequences, it will be secreted into the lumen of the oviduct and onto the yolk of an egg.

Some founders are germ-line founders. A germ-line founder is a founder that carries the transgene in genetic material of its germ-line tissue, and may also carry the transgene in oviduct magnum tubular gland cells that express the heterologous protein. Therefore, in accordance with the invention, the transgenic bird will have tubular gland cells expressing the heterologous protein and the offspring of the transgenic bird will also have oviduct magnum tubular gland cells that express the selected heterologous protein. (Alternatively, the offspring express a phenotype determined by expression of the exogenous gene in a specific tissue of the avian.)

The invention can be used to express, in large yields and at low cost, a wide range of desired proteins including those used as human and animal pharmaceuticals, diagnostics, and livestock feed additives. Proteins such as growth hormones, cytokines, structural proteins and enzymes including human growth hormone, interferon, lysozyme, and β-casein are examples of proteins which are desirably expressed in the oviduct and deposited in eggs according to the invention. Other possible proteins to be produced include, but are not limited to, albumin, α-1 antitrypsin, antithrombin III, collagen, factors VIII, IX, X (and the like), fibrinogen, hyaluronic acid, insulin, lactoferrin, protein C, erythropoietin (EPO), granulocyte colony-stimulating factor (G-CSF), granulocyte macrophage colony-stimulating factor (GM-CSF), tissue-type plasminogen activator (tPA), feed additive enzymes, somatotropin, and chymotrypsin. Immunoglobulins (shown, for example in Example 10 below) and genetically engineered antibodies, including immunotoxins which bind to surface antigens on human tumor cells and destroy them, can also be expressed for use as pharmaceuticals or diagnostics.

In various embodiments of the transgenic bird of the present invention, the expression of the transgene may be restricted to specific subsets of cells, tissues or developmental stages utilizing, for example, trans-acting factors acting on the transcriptional regulatory region operably linked to the polypeptide-encoding region of interest of the present invention and which control gene expression in the desired pattern. Tissue-specific regulatory sequences and conditional regulatory sequences can be used to control expression of the transgene in certain spatial patterns. Moreover, temporal patterns of expression can be provided by, for example, conditional recombination systems or prokaryotic transcriptional regulatory sequences.

The stably modified oviduct cells will express the heterologous polynucleotide and deposit the resulting polypeptide into the egg white of a laid egg. For this purpose, the expression vector will further comprise an oviduct-specific promoter such as ovalbumin or ovomucoid operably linked to the desired heterologous polynucleotide.

Another aspect of the present invention provides a method for the production in an avian of a heterologous protein capable of forming an antibody suitable for selectively binding an antigen. This method comprises a step of producing a transgenic avian incorporating at least one transgene, the transgene encoding at least one heterologous polypeptide selected from an immunoglobulin heavy chain variable region, an immunoglobulin heavy chain comprising a variable region and a constant region, an immunoglobulin light chain variable region, an immunoglobulin light chain comprising a variable region and a constant region, and a single-chain antibody comprising two peptide-linked immunoglobulin variable regions.

In one embodiment of this method, the isolated heterologous protein is an antibody capable of selectively binding to an antigen and which may be generated by combining a t least one immunoglobulin heavy chain variable region and at least one immunoglobulin light chain variable region, preferably cross-linked by at least one disulfide bridge. The combination of the two variable regions generates a binding site that binds an antigen using methods for antibody reconstitution that are well known in the art.

The present invention also encompasses immunoglobulin heavy and light chains, or variants or derivatives thereof, to be expressed in separate transgenic avians, and thereafter isolated from separate media including serum or eggs, each isolate comprising one or more distinct species of immunoglobulin polypeptide. The method may further comprise the step of combining a plurality of isolated heterologous immunoglobulin polypeptides, thereby producing an antibody capable of selectively binding to an antigen. In this embodiment, for instance, two or more individual transgenic avians may be generated wherein one transgenic produces serum or eggs having an immunoglobulin heavy chain variable region, or a polypeptide comprising such, expressed therein. A second transgenic animal, having a second transgene, produces serum or eggs having an immunoglobulin light chain variable region, or a polypeptide comprising such, expressed therein. The polypeptides from two or more transgenic animals may be isolated from their respective sera and eggs and combined in vitro to generate a binding site capable of binding an antigen.

The present invention is further illustrated by the following examples, which are provided by way of illustration and should not be construed as limiting the scope of the claims or the scope of the specification. The contents of all references, published patent applications and patents cited throughout the present application are hereby incorporated by reference in their entireties.

It will be apparent to those skilled in the art that various modifications, combinations, additions, deletions and variations can be made in the present invention without departing from the scope or spirit of the invention. For instance, features illustrated or described as part of one embodiment can be used in another embodiment to yield a still further embodiment. It is intended that the present invention covers such modifications, combinations, additions, deletions and variations as come within the scope of the appended claims and their equivalents.

### EXAMPLE 1

Generation of Transgenic Blastodermal Cells by EGFP Screening

a) Linearized targeting vector was prepared as follows:

i) Use supercoiled plasmid purified by cesium chloride banding [Sambrook, J., Fritsch, E. F., and Maniatis, T. (1989). Molecular Cloning: A Laboratory Manual, Second Edition (Cold Spring Harbor, N.Y.: Cold Spring Harbor Laboratory Press].

ii) In 1.5 milliliter eppendorf tube add 100 to 150 microgram plasmid (in a volume of less than 100 microliter).

iii) In the same tube adjust volume to 350 microliter with distilled, deionized water (ddH2O), add 40 microliter 10× restriction enzyme buffer and 200 units of restriction enzyme. Bring final volume to 400 microliter with ddH2O.

iv) Mix well and put in water bath at the right temperature for the restriction enzyme. Incubate overnight (16 to 18 hours)

v) Take 10 microliter of the digestion and check for complete digestion on a 0.7% ethidium bromide agarose gel.

vi) In each tube add 5 microliter of 20 milligram/milliliter proteinase K.

vii) Incubate at 37° C. for 30 to 60 minutes.

viii) To each tube add 400 microliter ice-cold PCI (phenol:chloroform:iso-amyl alcohol, 25:24:1) and vortex vigorously for 1 minute.

ix) Centrifuge at 14000 rpm (RCF: 20800×g) for 4 to 6 minutes.

x) Transfer the supernatant to a fresh tube.

xi) Add 400 microliter chloroform or sevag (chloroform:iso-amyl alcohol, 24:1) to each tube and vortex vigorously for 1 minute.

xii) Centrifuge at 14000 rpm for 4 to 6 minutes.

xiii) Aspirate the supernatant to a fresh tube.

xiv) In each tube add 80 microliter 3M pH 5.2 sodium acetate, mix well and then add 1 milliliter −20° C. 100% ethanol (Pharmco, cat# Ethanol 64-17-5).

xv) Mix well and look for appearance of DNA precipitate.

xvi) Remove the DNA precipitate with a 1 milliliter pipette tip and place into a fresh tube within 1-1.5 milliliter 70% ethanol.

xvii) Wash the pellet by gently shaking ten times and then spin for 30 seconds at 6000 rpm (RCF 3800×g).

xviii) Dry the pellet in a speed-vac for 4 minutes with medium heat.

xix) Add 100 microliter 0.1×TE pH 8.0 to the pellet, pipet up and down several times and let the pellet dissolve at room temperature.

xx) Measure OD 260 nm and calculate the DNA concentration.

xxi) This DNA is ready for transfection.

b) Feeder cells were prepared as follows:

i) Thaw passage 10 or 11 vial, plate into 1-2 10 cm plates, split 1-5 or 6 when confluent.

ii) On average, one 10 cm dish of confluent STOs cells is sufficient for one 24-well plate (5 10 cm dishes are sufficient for 4 6-well plates).

iii) Culture STO cells at 37° C., 5% CO₂ with STO medium until cells become confluent.

iv) Prepare sterilized 0.1% gelatin (Sigma, Cat.No. G-9391) solution.

v) In each well/24 well plate (Falcon, 3047) add 0.2 to 0.3 milliliters 0.1% gelatin solution (for 6 well, 0.962 ml gelatin per well was used).

vi) Incubate for 2 to 5 minutes at room temperature, then aspirate the solution and dry the plate in the hood.

vii) Treat cells with 10 microgram per milliliter mitomycin C in STO medium at 37° C., 5% CO₂ for 2.5 hours.

viii) Aspirate the medium and wash the cells with 5 milliliter PBS without Ca⁺⁺ and Mg⁺⁺ two times.

ix) Add 3 milliliter 0.05% trypsin-EDTA (0.02%) without Ca⁺⁺ and Mg⁺⁺ prewarmed to 37° C. to each 10 cm dish and incubate at 37° C. for 3 to 4 minutes.

x) Use pipette to dissociate the cells and transfer to 15 milliliter sterile tube containing 0.5 milliliter FBS.

xi) Wash the dish with 3 milliliter of STO medium prewarmed at 37° C. and combine with cells in 15 milliliter tube.

xii) Spin at 500 rpm (RCF 57×g) at room temperature for 5 minutes.

xiii) Aspirate the supernatant.

xiv) For each tube from a 10 cm dish of cells, add 6 milliliter STO medium prewarmed at 37° C. and resuspend the cells by gently pipetting for 20 times using 5 milliliter pipet.

xv) Add another 6 milliliter medium into the tube and resuspend very well by gently pipetting 10 times.

xvi) Aliquot 0.5 milliliter cell (2×10⁵ cell) suspension per well of a 24 well plate (for 6-well plate, 9.62×10⁵ cells or 2.4 mls per well).

xvii) 6-well or 24-well plates with treated STO cells can be used in 1 or 2 days.

xviii) Be sure to use fresh cells; in other words use the cells immediately after preparation.

c) Stage X Barred Rock blastodermal cells were prepared as follows:

i) Collect fertilized Barred Rock eggs within 48 hours of laying.

ii) Use 70% ethanol to clean the egg's shell.

iii) Crack the shell and open the egg.

iv) Remove egg white by transferring yolk to opposite half of shell, repeat several times to remove most of the egg white.

v) Put egg yolk with embryo disc facing up into a 10 cm petri dish.

vi) Use a Kim-wipe to gently remove egg white from the embryo disc.

vii) Place a paper (Whatman, filter paper 1, Cat. No. 1001 400) ring over the embryo.

viii) Use scissors to cut the membrane along the outside edge of the paper ring while gently lifting the ring/embryo with a pair of tweezers.

ix) Take the paper ring with the embryo and insert at a 45 degrees angle into a petri dish containing RT PBS-G solution (Gibco BRL Cat. No. 11500-030).

x) After ten embryo discs are collected, gently wash the yolk from the blastoderm disc using a Pasteur pipette under a stereo microscope.

xi) Cut the disc by a hair ring cutter. (a short piece of human hair is bent into a small loop and fastened to the narrow end of a Pasteur pipette with Parafilm).

xii) Remove the disc from the dish with a Pasteur pipette and place into a 15 milliliter sterile centrifuge tube (Fisher brand, Cat. No. 05-539-5) on ice.

xiii) Place 10 to 15 embryos per tube and let settle to the bottom (about 5 minutes).

xiv) Aspirate supernatant from the tube.

xv) Add 5 milliliter ice-cold PBS without Ca⁺⁺ and Mg⁺⁺, and gently pipette 4 to 5 times using a 5 ml pipette.

xvi) Incubate in ice for 5 to 7 minutes to allow the blastoderms to settle and aspirate the supernatant.

xvii) Add 3 milliliter ice cold 0.05% trypsin/0.02% ETDA to each tube and gently pipette 3 to 5 times using a 5 ml pipette.

xviii) Put the tube in ice and set timer for 10 minutes. After 5 minutes flick the tube by finger for 40 times and repeat again after another 5 minutes.

xix) Add 0.5 milliliter FBS and 3 to 5 milliliter BDC medium to each tube and gently pipette 5 to 7 times using a 5 ml pipette.

xx) Spin at 500 rpm (RCF 57×g) 4° C. for 5 minutes.

xxi) Aspirate the supernatant and add 2 milliliter ice cold BDC medium into each tube.

xxii) Resuspend the cells by gently pipetting 20 to 25 times by using 5 ml pipette.

xxiii) Take 10 microliters to determine the cell titer by hemacytometer ensuring 95% of all BDCs are single cells and not in clumps.

d) Linearized plasmids were transfected into BDCs by electroporation as follows (large scale, 0.4 cm cuvette).

i) Centrifuge the BDC suspension at 500 rpm (RCF 57×g), 4° C., 5 minutes.

ii) Resuspend BDCs to 3×10⁶/milliliter with PBS without Ca and Mg in a 15 milliliter centrifuge tube.

iii) Add linearized DNA at 50-100 microgram per 3×10⁶ BDCs and mix well.

iv) Incubate on ice for 10 minutes.

v) Aliquot 800 microliter of DNA-BDC mixture to a 0.4 cm Gene Pulser cuvette made by BIO-RAD

vi) Keep the cuvette on ice.

vii) Electroporation at 240V and 250uFD using Gene Pulser II made by BIO-RAD.

viii) Incubate the cuvette on ice for 20 minutes after electroporation.

ix) Add 1 milliliter ice-cold BDC medium into the cuvette and gently pipette several times by using 1 milliliter pipette.

x) Transfer the BDC suspension to a 50 milliliter tube and use 2 milliliter BDC medium to wash the cuvette and combine with the BDC suspension.

xi) Add the BDC medium to a concentration of 2.5-3×10⁵ cells per 2.5 milliliter.

xii) Aliquot 2.5 milliliter of cell suspension to each well of a 6 well plate with STO feeders treated with mitomycin C.

xiii) Culture the cells at 39.5° C., 5% CO₂.

xiv) Change the medium daily and replace with 2.5 milliliter fresh BDC medium, pre-warmed to 37° C., per well.

xv) After culture for two to ten days the colonies are ready for identification and harvest. Preferred time for harvest is four to ten days.

e) Transfection of linearized plasmids into BDCs by electroporation was done as follows: (small scale, 0.1 cm cuvette, one condition, two wells of 6-well plate).

i) Centrifuge the BDC suspension at 500 rpm (RCF 57×g), 4° C., 5 minutes.

ii) Resuspend BDCs to 3×10⁶/milliliter with PBS without Ca and Mg in a tube.

iii) Add linearized DNA at 50-100 microgram per 3×10⁶ BDCs and mix well; 5-10 ug per 3×10⁵ BDCs in 100 ul per well.

iv) Incubate on ice for 10 minutes.

v) Aliquot 100 microliter of DNA-BDC mixture to a 0.1 cm Gene Pulser cuvette made by BIO-RAD.

vi) Keep the cuvette on ice. (Just before electroporation, triturate several times with p200.)

vii) Electroporation at 240V and 25 uFD using Gene Pulser II made by BIO-RAD.

viii) Incubate the cuvette on ice for 20 minutes after electroporation.

ix) Add 0.125 milliliter ice-cold BDC medium into the cuvette and gently pipette several times by using 1 milliliter pipette.

x) Transfer the BDC suspension to a tube and use 0.25 milliliter BDC medium to wash the cuvette and combine with the BDC suspension.

xi) Add the BDC medium into the cell resuspend at concentration of 2.5-3×10⁵ cells per 2.5 milliliter.

xii) Aliquot 2.5 milliliter of cell suspension to each well of a 6 well plate with STO feeders treated with mitomycin C.

xiii) Culture the cells at 39.5° C., 5% CO₂.

xiv) Change the medium every day and replace with 2.5 milliliter fresh BDC medium, pre-warmed at 37° C., per well.

xv) After culture for two to ten days the colonies are ready for identification and harvest. Preferred time for harvest is four to ten days.

f) homogenous fluorescent green colonies were identified and harvested as follows:

i) This protocol is used when cells were transfected with plasmids containing the RSV promoter driving EGFP (Clontech).

ii) Under inverted microscope with FITC illumination [Olympus IX70, 100 W mercury lamp, HQ-FITC Band Pass Emission filter cube, exciter 480/40 nm, emission 535/50 nm, 20× phase contrast objective (total magnification is 2.5×10×20)], quickly screen CBC colonies.

iii) Select colonies in which all or most of the cells are fluorescing. All EGFP-positive cells within a colony should fluoresce at a similar intensity. If the fluorescence does not look like it is EGFP derived (an off-green or yellow-green color), check for auto-fluorescence using a TRITC filter (Olympus Modular B-MAX Filter cube, excitation 535/50 nm, emission 610/75 nm). A true EGFP-positive cell should not fluoresce under the TRITC filter.

iv) Limit UV exposure of each colony to 30 seconds or less.

v) Close the UV shutter and switch to visible light. Use a fine glass needle to cut around the desired colony. Fisherbrand Disposable Micropipets (cat no. 21-164-2G, 50 microliters, borosilicate glass) which have been pulled (David Kopt Instruments Needle Puller, Model 700C, heater set at 50 for 20 amp reading, solenoid set at 10) are used.

vi) Pick up marked colonies under visible light using a fine glass needle (see step v, above) attached via rubber tubing to a 3 cc syringe. The end of the needle is broken off such the internal diameter is wide enough for passage of CBC colonies.

vii) Put colonies into an Eppendorf tube with 100 microliter ice-cold BDC medium on ice.

viii) Collect 100 to 150 colonies for each tube.

g) Production of transgenic chickens was done as follows:

i) Centrifuge the colonies at 500 rpm (RCF 57×g) at room temperature.

ii) Aspirate supernatant carefully.

iii) Add 1 milliliter 37° C. PBS without Ca and Mg with 0.02% EDTA.

iv) Incubate at room temperature for 10 minutes.

v) Centrifuge at 500 rpm (RCF 57×g) at room temperature.

vi) Aspirate supernatant carefully.

vii) Add 0.3 milliliter 37° C. 0.05% trypsin without Ca and Mg with 0.02% EDTA (Gibco BRL 25300-054).

viii) Put the tube into 37° C., 5% CO₂ incubator for 5 minutes.

ix) Add 0.1 milliliter FBS and 0.5 milliliter BDC medium (room temperature) into the tube.

x) Mix gently up and down by using 1 milliliter Pipetman tip.

xi) Spin at 500 rpm (RCF 57×g) for 5 minutes at 4° C.

xii) Aspirate the supernatant.

xiii) Resuspend the cells in 50 to 100 microliter ice-cold BDC medium.

xiv) Prepare and inject White Leghorn embryos according the method described in U.S. Pat. No. 5,897,998, Speksnijder. et al. Eggs have been gamma irradiated at 600 rads.

xv) Inject into each White Leghorn embryo an average of 1000 CBCs (the equivalent of 10 colonies).

### EXAMPLE 2

Production of Transgenic Whole Embryo Fibroblasts by Puromycin Selection

Large scale electroporation: high throughput screening of a targeted gene.

i) From 30 to 40 10 cm plates of WEFs, passage 3, freshly isolated (not from frozen stock), cells are nearly confluent, still fair number of non WEF cells in the culture.

ii) Wash plates 1× with sterile 1× PBS. Add 2 mls of 0.05% trypsin +0.02% EDTA. Incubated for 5 min at 37° C., 5% CO2.

iii) Add 4 mls of WEF media to each plate. Mix and transfer suspension to 50 ml conical tubes (5-6 plates per tube).

iv) Pellet cells at 500 rpm, 4oC for 5 minutes.

v) Resuspend each pellet in 5 mls CMF-PBS and combine pellets. Bring final volume to 45 mls with CMF-PBS.

vi) Repellet cells.

vii) Resuspend pellet in 825 ul CMF-PBS.

viii) Count cells.

ix) To an eppendorf tube, add 5×106 cells, qs to 800 ul with CMF-PBS.

x) Add 50 ug linearized RSV-pur vector to each tube.

xi) Incubate on ice for 10 min.

xii) Transfer tube to 0.4 cm electroporation cuvette.

xiii) Electroporate at 240 V, 250 uF, time constant should be 6.6 to 6.9 mS.

xiv) Incubate on ice for 10 min.

xv) Transfer cells to 4.2 ml of WEF media and rinse out the cuvette to get remaining cells.

xvi) Expect ˜1000 cells/ul.

xvii) To a well of a gelatinized 6 well plate, add 200 ul of the suspension plus 2 mls of WEF media.

xviii) Culture at 37oC, 5% CO2.

xix) Add puromycin to 0.5 ug/ml 24 or 48 hours later.

xx) Expect ˜25 colonies per well, which will grow to confluency within two weeks.

Small scale electroporation was done as follows: (similar to a large scale except for the following differences):

i) Scale down the number of plates harvested accordingly.

ii) To an eppendorf tube, add 2×105 cells, qs to 100 ul with CMF-PBS.

iii) Add 5-10 ug linearized RSV-pur vector to each tube.

iv) Incubate on ice for 10 min.

v) Transfer tube to 0.1 cm electroporation cuvette.

vi) Electroporate at 240 V, 25 uF, time constant should be ˜0.5 mS.

vii) Incubate on ice for 10 min.

viii) Transfer cells to 2.0 ml of WEF media.

ix) To a well of a gelatinized 6 well plate or a 6 or 10 cm plate, add the cells.

x) Expect ˜20 well separated colonies if plated in a 6 or 10 cm plate.

Production of transgenic blastodermal cells by puromycin selection was done as follows:

Chicken embryo extract (CEE) was prepared as follows:

i) Incubate fertilized Barred Plymouth Rock chicken eggs for 7 days.

ii) Crack the eggs and cut the embryo and bring it into a 50 ml centrifuge tube with 10 ml cold PBS without Ca and Mg contained 0.15M NaCl.

iii) Collect 10 embryos for each tube on ice.

iv) Pour whole embryos into a pre-chilled blander vassal (Waring blander 25 mm size).

v) Blend for 10 seconds and take a little sample and check under microscope to find if the tissues become single cells (70-80%). If not, continue to blend several seconds until reaching above 70% single cells.

vi) Pour the homogenate into a fresh 50 ml centrifuge tube pre-chilled on ice.

vii) Freeze the homogenate in liquid nitrogen and thaw it at 37° C. water bath for three times.

viii) Transfer the homogenate into a centrifuge tube with screw cap pre-chilled on ice by pouring it.

ix) Balance the centrifuge tubes and centrifuge them at □C, 20000 g for 30 minuets.

x) Take the supernatant into a fresh 50 ml centrifuge tube on ice by pouring.

xi) Aliquot it to 1.5 ml eppendorf tubes at 1 chick embryo per tube.

xii) Freeze these tubes in −70° C. refrigerator.

BDC medium containing chicken embryo extract was prepared as follows:.

i) Filter the CEE by using Acrodic 25 mm syringe filter, with 0.45 um HT Tuffryn membrane, Gelman Laboratory (Cat. 2004-03). This step is optional but is preferred.

ii) Take the CEE from −70° C. refrigerator and thaw them on ice.

iii) Calculate how many milliliters equal to 1 chick embryo after filtering.

iv) Add the CEE into the BDC medium at 1 chick embryo per 40 ml BDC medium.

Linearized targeting vector was prepared as described in Example 1. Transfection of linearized plasmids into BDCs was done by electroporation as describe in Example 1.

Puromycin resistant CBC colonies were selected for as follows:

i) After transfection of BDCs with targeting vector, the transfected BDCs are cultured in BDC medium with CEE. 24 hours later, the BDC-CEE medium contained 0.5 ug puromycin per ml replaces the old medium.

ii) Change the medium with BDC-CEE-Puromycin medium every day.

iii) Most CBCs will die after several days' selection.

iv) At the day 7 to day 8 the puromycin resistant CBC colonies will show up.

v) Screen the CBC colony by naked eye and label it with mark pen.

vi) Further identify the CBC colony under microscope.

vii) Count the puromycin resistant CBC colonies.

Puromycin resistant CBC colonies were cultured as follows:

i) Cut the CBC colonies with sterile glass needle.

i) Pick up the CBC colonies with sterile glass needle.

iii) Dissociate the CBC colonies into single cells or small clumps by pipeting up and down for 30 to 50 times at 100 ul volume.

iv) Spread these CBC cells and clumps on to a well/24 well plate contained STO cells treated by mitomycin C.

v) Culture these CBCs for 4 to 6 days and change the medium with BDC-CEE-Puromycin medium every day.

vi) Dissociate the CBC colonies by pipetting up and down for 50 times at 500 ul volume.

vii) Transfer these CBCs on to a fresh well/24 well plate contained S to cells treated with mitomycin C.

viii) Culture the CBCs for about 6 days.

ix) Many CBC colonies will show up, the cell confluence will go to 40-100%.

x) Harvest the CBCs by trypsin method as standard.

Single puromycin resistant CBC colonies were cultured as follows:

i) Identify the puromycin resistant CBC colony.

ii) Cut and pick up the colony with sterile glass needle.

iii) Transfer the single CBC colony into a well of 96 well plate contained Sto cells treated with mitomycin C.

iv) Dissociate the colony by pipeting up and down 30 times at 70 ul volume.

v) Transfer whole dissociates to fresh well of 96 well plate contained Sto cells treated with mitomycin C.

vi) Wash with 100 ul BDC-CEE-Puromycin medium and transfer it to same well.

vii) Culture it at 39.5° C., 5% CO2 incubator for 7 to 9 days.

viii) Check all wells and discard differentiated one.

ix) Dissociate the CBC colonies in each well as above method.

x) Sub-culture each clone to 1-3 wells/96 well plate contained STO cells treated with mitomycin C according to how many new CBC colonies in each well.

xi) Culture it at 39.5° C., 5% CO2 incubator for 5-7 days.

xii) Dissociate again.

xiii) Sub-culture each clone in 1 well/24 well plate or 2-3 well/96 well plate.

xiv) After passage 3 or passage 4 the CBCs reach above 40% confluent at a well/24 well plate, harvest the CBCs by trypsin method and carry out injection to make chimera chicken as described in step 1f.

Confirmation that homogenous green fluorescent CBC colonies are stably transformed cells was done as follows:

(This method employs some of the techniques described in which individual EGFP positive colonies are picked and passaged several times.)

i) Transfect the BDCs with the linearized vector contained CMV-EGFG marker gene.

ii) Culture these BDCs with BDC-CEE medium for 4 days.

iii) Identify and pick up homogenous green fluorescent CBC colonies.

iv) Dissociate these colonies with above method.

v) Sub-culture the CBCs in the fresh well/24 well plate for 3-5 days.

vi) Check the CBC colonies under microscope with UV light.

vii) Find non-fluorescent and homogenous green fluorescent colonies.

viii) Pick up homogenous green fluorescent colonies.

ix) Dissociate these CBC colonies and sub-culture them for 3-5 days.

x) Check the fluorescence of the CBC colonies, and they all should be homogenous green fluorescent colonies.

xi) Continue culture these cells passage by passage and obtain enough cells (1×10 cm dish confluent cells should be enough) to extract DNA for southern blot.

### EXAMPLE 3

Culture Medium and Reagents

a) BDC culture medium

i) 409.5 milliliter DMEM with high glucose, L-glutamine, sodium pyruvate, pyridoxine hydrochloride (GibcoBRL Cat. No.: 11995-065).

ii) 5 milliliter Men non-essential amino acids solution 10 mM 100× (GibcoBRL Cat. No.: 11140-050).

iii) 5 milliliter Penicillin-streptomycin 5000 U/milliliter each (GibcoBRL Cat. No.: 15070-063).

iv) 5 milliliter L-Glutamine 200 mM 100× (GibcoBRL Cat. No.: 25030-081).

v) 75 milliliter Fetal bovine serum (Hyclone Cat. No.: Sh30071.03).

vi) 0.5 milliliter β-mercaptoethanol 11.2 mM 1000× (Sigma Cat. No.: M7522).

vii) Final volume is 500 milliliter.

b) STO, SNL and MEF culture medium

i) 435 milliliter DMEM with high glucose, L-glutamine, sodium pyruvate, pyridoxine hydrochloride (GibcoBRL Cat. No.: 11995-065).

ii) 5 milliliter Men non-essential amino acids solution 10 mM 100× (GibcoBRL Cat. No.: 11140-050).

iii) 5 milliliter Penicillin-streptomycin 5000 U/milliliter each (GibcoBRL Cat. No.: 15070-063).

iv) 5 milliliter L-Glutamine 200 mM 100× (GibcoBRL Cat. No.: 25030-081).

v) 50 milliliter fetal bovine serum (Hyclone Cat. No.: Sh30071.03).

vi) Final volume is 500 milliliter.

### EXAMPLE 4

Screening for Blastodermal Cells with Integrated Vector

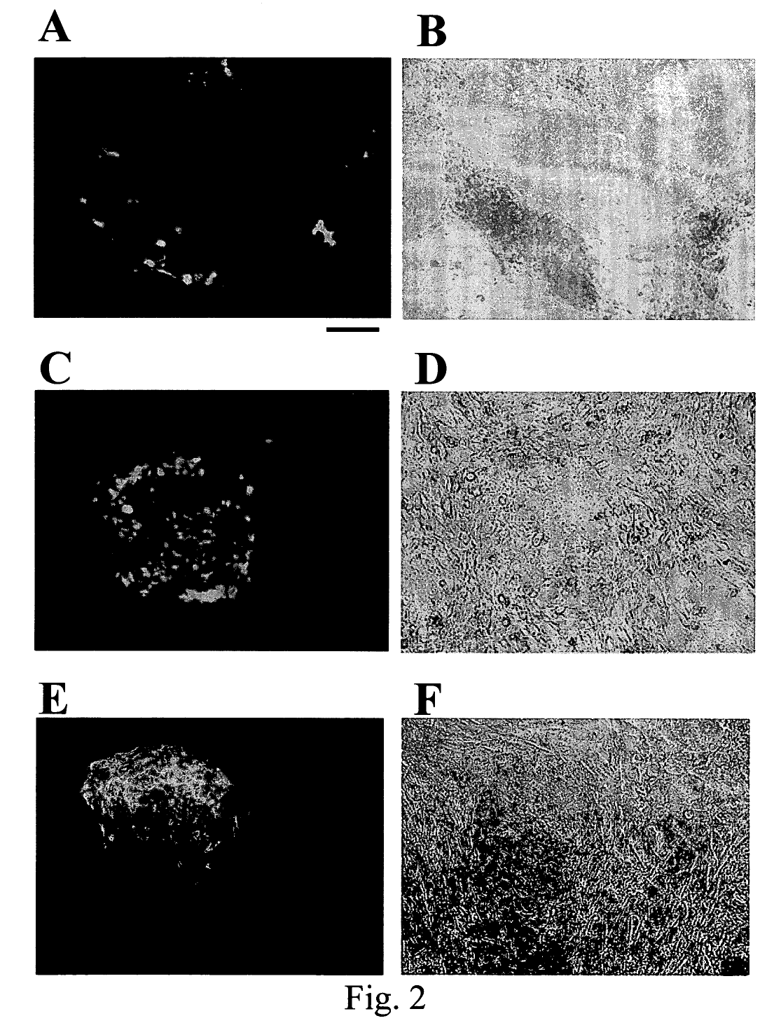

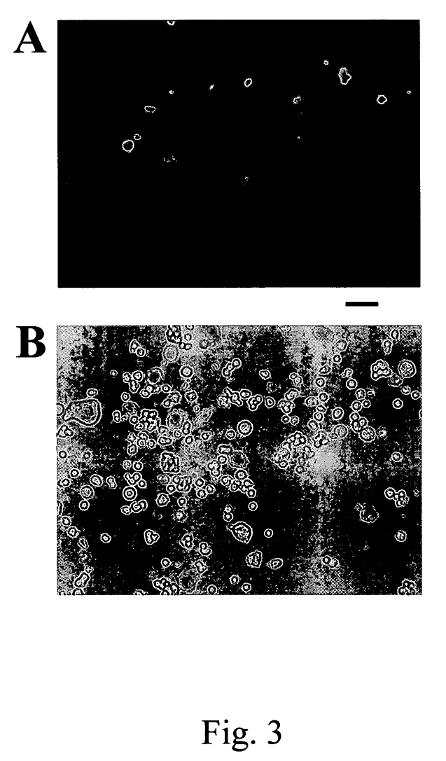

Typically, hundreds of chicken stage X embryos were collected from fertilized Barred Rock eggs and trypsinized. Approximately 6.0×10⁶ BDCs were washed with PBS without Ca and Mg and were mixed with several hundred micrograms of linearized pOVTV7.4/0.875-IFN-RSV-EGFP vector (FIG. 1) and electroporated at 240 voltage and 500 uFD capacity. The cells were spread onto STOs feeder treated with mitomycin C and cultured at 39.5° C., 5% CO2 with BDC medium. The media was changed each day for four days and the green fluorescent CBC colonies were identified under a microscope with UV light. Colonies with green fluorescence can be seen in FIG. 2. Several hundred fluorescence colonies were cut and picked up by using sterilized glass needles. These colonies were trypsinized and the cells were counted. The CBCs were checked under UV light for green fluorescence in the cell, (FIG. 3). About 20% of all cells were fluorescent. The CBCs were injected into White Leghorn stage X embryos which were irradiated at 600 Rads. The eggs were hatched by standard method. Table 1 shows some experiment results.

TABLE 1

| | Chicken | | | | | | |
|---|---|---|---|---|---|---|---|
| | stage × | | GFP | | | | |
| | embryos | BDCs × | colonies | CBCs per | Embryos | Chicks | composite |
| Experiment | collected | (10⁶) | harvested* | Injections | injected | hatched | chicks |
| May 12, 2000 | 171 | 6.7 | 365 | 4-5 × 10³ | 36 | 17 | 1 |
| May 19, 2000 | 194 | 9.1 | 302 | 2.5 × 10³ | 36 | 12 | 1 |
| Jun. 2, 2000 | 154 | 6.6 | 314 | 3 × 10³ | 30 | 30 | 1 |
| Jun. 9, 2000 | 185 | 7.2 | 337 | 1-1.5 × 10³ | 28 | 15 | 0 |
| Jun. 16, 2000 | 122 | 4.35 | 246 | 2.1 × 10³ | 26 | 7 | 1 |
| Jun. 23, 2000 | 146 | 6.1 | 358 | 1.4 × 10³ | 25 | 15 | 0 |
| Jul. 7, 2000 | 104 | 3.79 | 239 | 4.5 × 10³ | 24 | 13 | 0 |
| Jul. 28, 2000 | 68 | 2.7 | 121 | 4.3 × 10³ | 17 | 14 | 2 |
| Aug. 4, 2000 | 137 | 6.8 | 298 | 3.8 × 10³ | 22 | 12 | 0 |

*Typically only 20-30 homogenous EGFP+ CBC colonies were islolated. The remainder of the colonies were partially EGFP+.

### EXAMPLE 5

Optimization of Drug Selection in Avian Cells Using Whole Embryo Fibroblasts (WEFs).

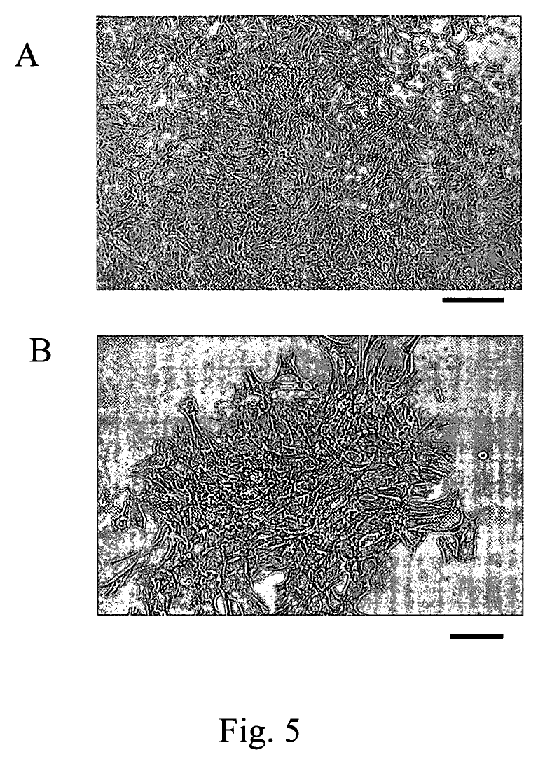

Puromycin selection in avian embryonic cells was first developed using WEFs. After testing blasticidin and neomycin, it was found the puromycin worked well in killing non-transformed cells while allowing the growth of healthy transformed WEFs (FIG. 5). A second advance made in the WEF system was that use of the RSV promoter produced many more puromycin resistant colonies than the CMV promoter (see Table 2). With the CMV promoter the average transformation efficiency (number of colonies divided by the total number of cells electroporated) was 0.00025% while with the RSV promoter it was 0.00825%.

TABLE 2

| | total no. of cells | no. of | Average % | |
|---|---|---|---|---|
| Promoter | electroporated | experiments | efficiency | std dev |
| CMV | 13200000 | 8 | 0.00025 | 0.00017 |
| RSV | 18640000 | 28 | 0.00825 | 0.00694 |

WEFs, while not able to form composites, could be used as donors for nuclear transfer. Additional experiments indicate that WEFs could also be useful to create cells with a targeted gene (data not shown).

### EXAMPLE 6

Puromycin Selection of Blastodermal Cells.

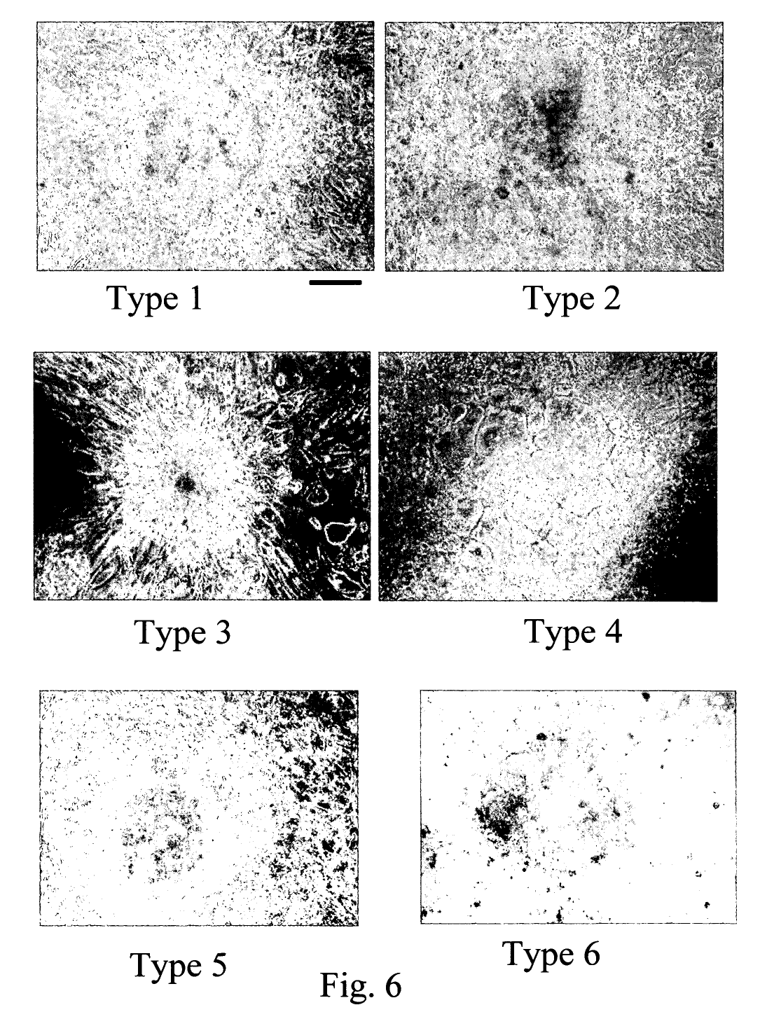

Typically, hundreds of chicken stage X embryos were collected from fertilized Barred Rock chicken and trypsinized. Several million BDCs were washed with PBS without Ca and Mg and mixed with several hundred micrograms of linearized pOVTV7.4/0.875-IFN-RSV-pac (FIG. 1) and electroporated at 240 voltage and 500 uFD capacity. The cells were spread onto STOs feeder treated with mitomycin C and cultured at 39.5° C., 5% CO2 with BDC-CEE medium. After 24 hours the BDC-CEE medium with 0.5 ug per ml puromycin was applied to the cultures. The medium was changed everyday. The puromycin selection continued for 7 to 8 days until the CBC colonies could be seen by the naked eye (FIG. 6).

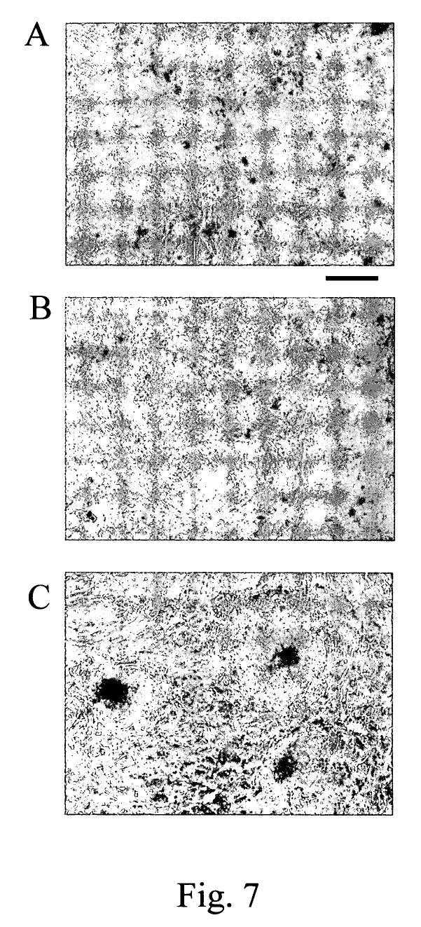

The CBC colonies were labeled and re-checked under microscope. These CBC colonies were cut and picked up with sterile glass needle. All of the colonies collected were mixed and then dissociated by the pipetting method and then cultured in a well of a 24 well plate with fresh STOs. After 4 to 6 days of culture, many new CBC colonies formed (FIG. 7). These CBC colonies were then dissociated by pipeting method and sub-cultured in a well 24 well plate with fresh STOs in it. When the CBC colonies reached 40-90% confluency the cells were trypsinized, counted and tested for viability with trypan blue stain. The CBCs were injected into chicken stage X embryos, which were irradiated at 600 Rad. The eggs were hatched by standard method. Table 3 shows some experiment results.

TABLE 3

| | | | | | | CBCs | | | |
|---|---|---|---|---|---|---|---|---|---|
| | | | | | | injected | | | |
| | | | Puromycin | | | per | | | |
| | Embryos | BDCs × | resistant | Transformation | | embryo × | Embryos | Chicks | Composite |
| Experiment | collected | 10⁶ | colonies | efficiency | passages | 10⁴ | injected | hatched | chicken |
| Jul. 19, 2000 | 105 | 3.8 | 70 | 0.00184% | P2 90% conf. | 1.35 | 43 | 7 | 5 |
| Aug. 2, 2000 | 155 | 5.85 | 73 | 0.00125% | p2, 90% conf. | 2.28 | 38 | 16 | 5 |
| Aug. 11, 2000 | 114 | 4.5 | 33 | 0.00073% | p2 40% conf. | 2.3 | 20 | 2 | 2 |
| Nov. 1, 2000 | 197 | 8.3 | 47 | 0.00057% | p2, 40% conf. | 2.1 | 36 | 10 | 4 |
| Jan. 10, 2001 | 181 | 6.7 | 53 | 0.00079% | p3, 70% conf. | 1.32 | 39 | 2 | 0 |
| Jan. 17, 2001 | 170 | 6.6 | 65 | 0.00098% | p2 | 1.3 | 42 | 8 | 4 |
| Feb. 9, 2001 | 182 | 6.4 | 66 | 0.00103% | p2, 30% conf. | 1.1 | 29 | 6 | 1 |
| Feb. 16, 2001 | 165 | 6.04 | 70 | 0.00116% | p2, 40% conf. | 1.2 | 36 | 12 | 1 |

As can be seen in Table 3, the transformation efficiencies ranged in one in 50,000 to one in 200,000 electroporated blastodermal cells. It should be of note that in earlier studies with the CMV promoter, none or very few colonies were produced (data not shown).

TABLE 4

| | | No. of | Feather | No. of blood | sperm DNA |
|---|---|---|---|---|---|
| Marker | Passage | birds | composites+ | composites+ | composites+ |
| EGFP | 0 | 189 | 8 | 0 | 0 |
| Pac | 2 to 3 | 91 | 28 | 8 | 0 |

Table 4 summarizes the total number of composites produced with either EGFP screening or pac selection. The ability to produce feather composites and birds that carried the transgene in their blood DNA (as determine by Taqman with the neo probe/primer set), was poor with EGFP and much better with pac. This was because many more colonies could be produced with puromycin selection. Furthermore, individual or groups of puromycin resistant colonies could be passaged, and thus amplified, so that there was an ample number of cells to inject into recipient embryos.

TABLE 5

| | | | | Feather |
|---|---|---|---|---|
| | | | % of blood | chimerism (% of |
| | Band | Hatch | cells with | feathers that are |
| CBC type | No. | date | transgene | black). |
| Passage 2 from mixed colony | 7783 | Sep-00 | 3.3 +/− 1.8 | 95% |
| Passage 2 from mixed colony | 8274 | Oct-00 | 0.6 +/− 1.3 | 90% |
| Passage 3 from single colony | 9618 | Nov-00 | 0.0 +/− 0.0 | 0 |
| Passage 4 from single colony | 9954 | Nov-00 | 14.95 +/− 1.1 | 30% |
| Passage 4 from single colony | 9968 | Nov-00 | 0.9 +/− 0.3 | 0% |
| Passage 2 from mixed colony | A102 | Dec-00 | 2.4 +/− 0.3 | 5% |
| Passage 2 from mixed colony | A170 | Dec-00 | 12.4 +/− 6.0 | 85% |
| Passage 2 from mixed colony | A171 | Dec-00 | 4.8 +/− 4.5 | 50% |
| Passage 2 from mixed colony | A176 | Dec-00 | 0.6 +/− 0.9 | 0% |

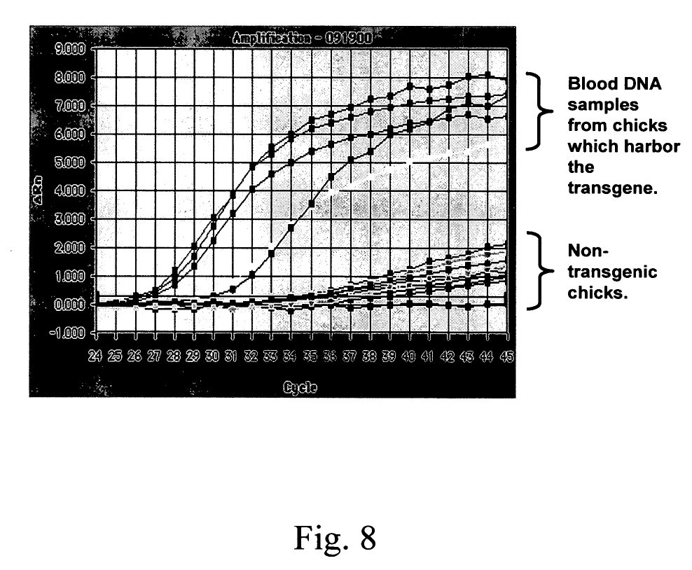

Table 5 shows data from some of the individual birds produced with pac. The % of blood cells with the transgene was performed with the quantitative Taqman assay using the neomycin primer/probe set (FIG. 8). [The neomycin sequence used is a ˜70 bp sequence that was cloned into the 3′ end of the polyadenylation region of the pac or EGFP expression cassette.] The data indicate that the cells are pluripotent in that they can contribute to at least several tissues. They are able to contribute to erythrocytes as evidenced by the presence of the transgene in the blood DNA. And they can contribute to melanocytes as evidenced by the positive feather chimerism.

### EXAMPLE 7

Culture of Single CBC Colonies

Of the puromycin resistant CBC colonies, six phenotypes based on morphology were recognized and characterized. The morphologies of each colony are represented in FIG. 6.

To further characterize the colony morphologies, single colonies of each type were picked and passaged. Only types 1, 2 and 4 could be passaged efficiently (Table 6). When cells from the other types of colonies were passaged, a very small percentage (<5%) would grow. The resulting culture would be at a very low density and would never reach a density that allowed harvesting for composite production or further passaging. Table 6 shows the results of the experiment.

TABLE 6
Passaging of single puromycin resistant CBC colonies culture.

| | | | | Cells | | | |
|---|---|---|---|---|---|---|---|
| | | | | injected | | | |
| | | At passage 1, | At passage 4, | per | | | |
| Colony | Clone | number of the | confluency of | embryos × | Embryos | Chicks | Composite |
| type | no. | colonies. | culture. | 10⁴ | injected | hatched | chickens |
| 1 | 1 | 1 | 60% confluent | 1.5 | 19 | 4 | 3 |
| | | (very large colony) | | | | | |
| 2 | 1 | 12 | 95% confluent | 1.9 | 26 | 1 | 0 |
| 2 | 17 | 10 | 80% confluent | 2 | 26 | 4 | 4 |
| 2 | 18 | 6 | 90% confluent | 2.1 | 23 | 4 | 3 |
| 2 | 19 | 12 | 100% confluent | 1.2 | 24 | 5 | 1 |
| 4 | 3 | 9 | 15% confluent | 0.68 | 22 | 3 | 0 |
| 4 | 5 | 14 | 30% confluent | 1.8 | 23 | 2 | 2 |

As can be seen in Table 6, at passage 1 there were a few colonies produced from single colony. By passage 4, there were hundreds to thousands of colonies such that most cultures became confluent if not passaged again. In this experiment, the mixture of CBC colonies and STOs were harvested at passage 4 injected into stage X embryos. The number of CBCs could be determined in presence of STOs because of the size differential between the two (see FIG. 3). The efficiency of production of composites with black feathers was very good, indicating that pluripotency can be maintained during the culture of single colonies. It should be of note that while we observed distinct morphologies prior to the first passage, most colonies after passage 1 or 2 exhibited the type 2 morphology (FIG. 7).

It should be of note that without the addition of CEE, groups of colonies of CBCs can be passaged but we observed that fewer colonies were obtained after the first passage than were observed in the primary culture. We were able to harvest a single colony without CEE, but we were not able to expand the colony into a sufficient number of colonies for maintenance or analysis of the colony.

### EXAMPLE 8

Confirmation of Stable Integration of the Transgene in Puromycin Resistant CBCs

In order to confirm that the transgenes were integrated and the nature of the integrations, single puromycin resistant CBC colonies were passaged and DNA extracted. Eight puromycin resistant CBC single colonies were cultured and passaged until a sufficient number of cells was available for DNA extraction. The DNA was extracted by a SDS/proteinase K digestion method (described in Maniatis for eukaryotic cells). DNA from non-transgenic CBCs cultured on STOs as well as DNA from WEFs were used a controls. To confirm integration of a transgene, it is convenient to digest the DNA that cuts the transgene once, in this case BamH I. The Southern blot is probed twice with probes that are unique to the transgene (non-avian) and are on both sides of the BamH I site. In this case we used probes complementary to the IFN and pac coding sequences.

Although we are using a vector, pOVTV-7.4/0.785-IFNMM-RSV-pac (EGFP), that was designed to recombine with the endogenous ovalbumin gene, we expected to isolate colonies that harbored a random insertion but none with a targeted gene. This is because the frequency of random insertion events in other manimalian systems is much higher (100-1000 fold) than that of homologous recombinations.

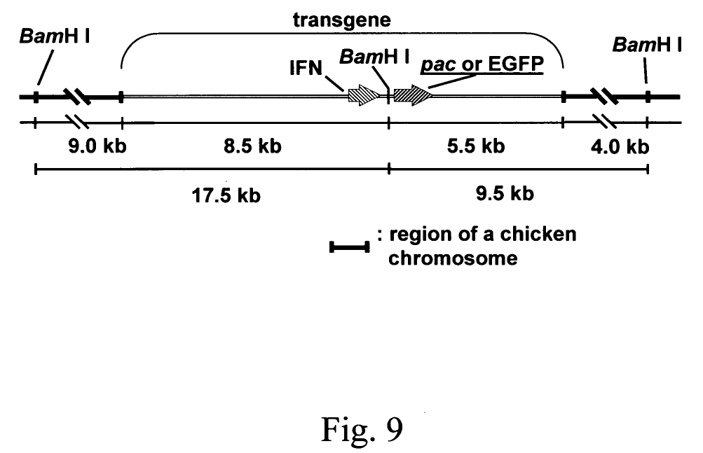

Integration of the transgene results in junction fragments due to the internal BamH I site and the sites that flank the transgene (FIG. 9). Each colony should have a uniquely-sized pair of junction fragments, depending of the spacing of genomic BamH I sites at the site of integration, which is random. Thus, integration is confirmed by making the following observations. First the bands detected by each probe are larger than the fragments of the linearized transgene. If not the transgene has either not integrated or has integrated but has undergone a deletion. Second, the bands detected by the two different probes should be of different lengths. If not the transgene is persisting episomally. Third, of the colonies that meet the above requirements, each colony should exhibit junction fragments of different sizes since each colony represents an integration into a different region of the chicken genome.

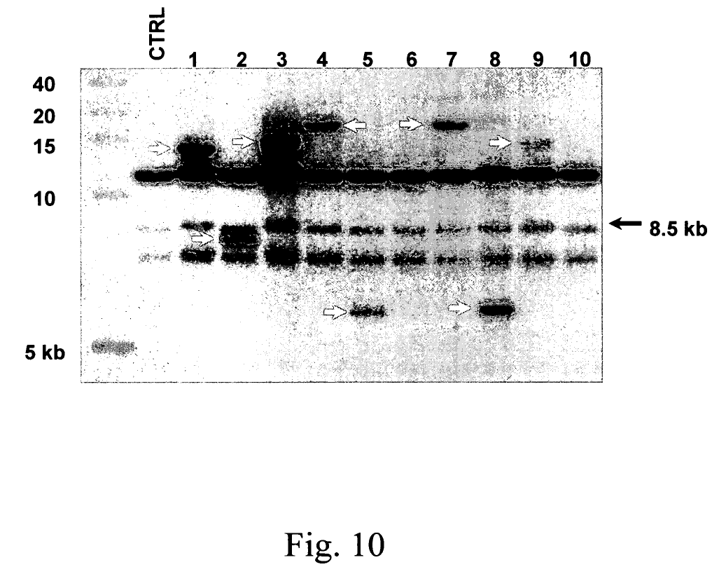

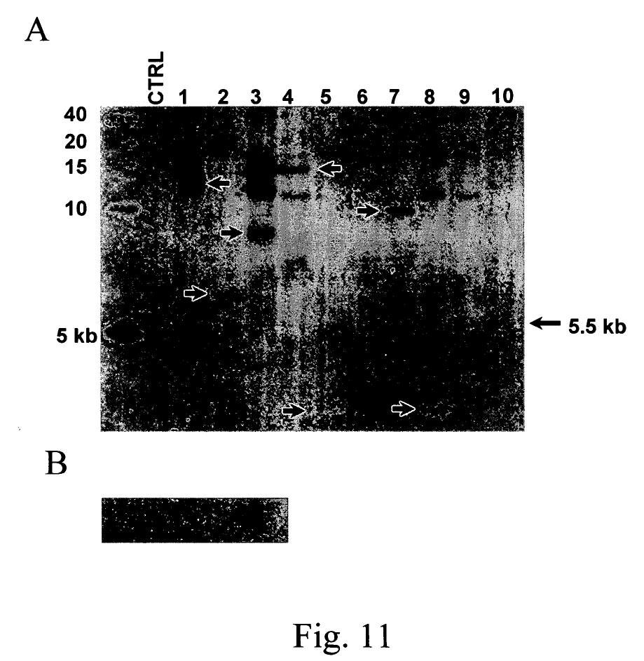

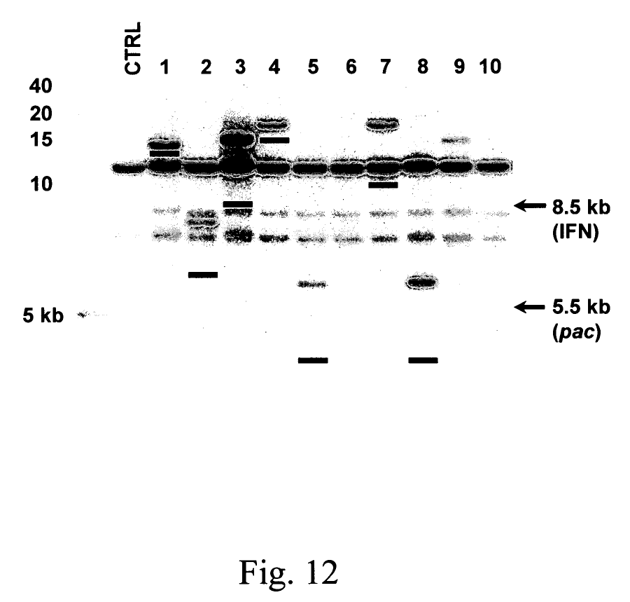

FIG. 9 outlines an example of an integrated pOVTV-7.4/0.785-IFNMM-RSV-pac transgene that integrated between two BamH I sites that were, prior to integration, 13 kb apart. In a Southern blot analysis of this example, a IFN probe should detect a BamH I-digested fragment of 17.5 kb and a pac probe should detect a fragment of 9.5 kb. This example is based on the results of a colony's Southern blot data displayed in FIGS. 10-11, lane 7. FIG. 12 is the same Southern blot shown in FIG. 10 and with the position of bands detected by the pac probe superimposed in order to demonstrate that each probe detected unique fragments.

The colonies in lanes 1, 3, 4 and 7 also fit the criteria for an integrated transgene. The colonies in lanes 5 and 8 appear to have had deletions on both sides of the transgene as both teh IFN and pac probes detected fragments that were shorter than the original transgene. The colonies in lanes 5 and 8 could be derived from the same original colony as both sides of the transgene are the same size. It is unclear how this could have occurred. The colony in lane2 had a deletion in the IFN arm.

### EXAMPLE 7

Confirmation of Transgene Integration into Chicken Genome

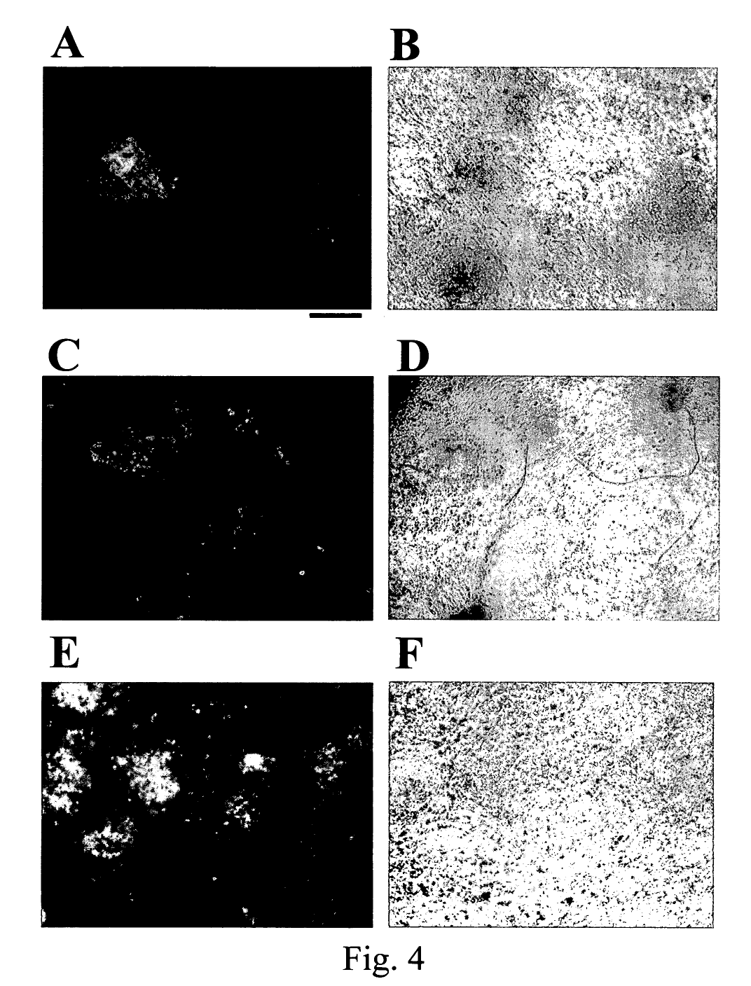

In order to determine whether the transgene had integrated into the chicken genome, 34 green fluorescent CBC colonies were picked up after transfection of BDCs with linearized pOVTV7.4/0.875-IFN-RSV-EGFP and cultured for 96 hours (this is defined as the primary culture). These colonies were dissociated by pipeting method and cultured in a well of 24 well-plate contained STOs and using BDC-CEE medium (see Methods section). After three days culture about 50% of the resulting colonies exhibited green fluorescence in whole colony (FIG. 4A, B)(this is defined as passage one). Others had no green fluorescence. Several undifferentiated homogenous green fluorescent colonies were picked up from the passage one culture, dissociated and cultured on STOs with BDC-CEE medium. All colonies exhibited homogenous green fluorescence in this and subsequent passages (FIG. 4C, D, E, F). The homogenous green fluorescent colonies were continuously cultured to passage 11 on the 10 centimeter gelatin dishes and the DNA was extracted for southern blot analysis (FIG. 10, lane 9). A single band of 13 kb was detected in Bam HI digested genomic DNA by the IFN probe. With integration, a band larger than 8.5 kb would be expected. Therefore, confirmation

### EXAMPLE 8

Cell Culture Protocols

Cell Lines

Certain standard mouse embryo stem cell culture protocols keep the ES cells undifferentiated by culturing the ES cells on the embryo fibroblast cell layer (feeder layer). STOs and SNL cell lines are often used as the feeder to culture the ES cells for gene targeting or knock out. In order to culture chicken blastodermal cells and keep them undifferentiated, several cell lines were tested as feeders. WEF and CEF cells come from different stage chicken embryos. They are all fibroblast cells. QOF cells are from quail embryos and it is similar with WEF. The BDCs were cultured on them for 4 to 6 days. The CBC colonies were checked under microscope. The results show that they are dispersed and un-intact. Although the CBCs proliferated very well on these feeders and from the CBCs, composite chickens were produced. The feeders cannot be used as feeders to culture the BDCs because the intact colony is needed for cloning. STO and SNL cell lines are transformed mouse fibroblast cells which can be cultured indefinitly. SNL cell line has Neo gene in its chromosome so it can be used directly as feeder for screening transformed colonies for integrated targeting vector with Neo gene as a marker gene in it. These two cell lines were tested for BDCs culture. The colonies of CBCs were checked and they are intact and have clear boundary with the feeder after four days culture. The CBCs were injected into chicken stage X embryos. The composite chicken can be produced from these injections. Other cells like MEFs from 14.5 days mouse embryos and BRL from rat liver were also tested for BDCs culture. CBCs colonies on the MEF will gradually differentiate into fibroblast cells and the colonies look like smaller and smaller after three days culture. Most BDCs can not seed on the BRL feeder and these BDCs fuse together to make some bigger balls. Very less BDCs form CBC colonies. The GC (granulosa cells from chicken fertilized egg) cells were also tested as the feeder, and CBCs produced yielded a very low percentage of composites. STOs are the best feeder for culturing BDCs.

Culture Temperature

Birds have different body temperature from mammalian animals. The BDCs were cultured at different temperatures from 37° C., 39.5° C. to 41.5° C. The proliferation was measured according to the colonies number and the colony volume or size. BDCs cultured at 41.5° C. appeared to have the greatest yield of colonies. At 39.5° C. the yield was lower than at 41.5° C.; but remarkably higher than the yield at 37° C. in number and size of the colonies. The BDCs were also tested on the different feeders and different temperature at same time. Feeders from birds gave the same result as above. Feeders from mammalian animals died very quickly when they were cultured at 41.5° C. Among these feeders STOs and SNL can survive for eight days to ten days, which are good enough for drug selection at relative high growth speed and big colony size. MEF and BRL may die after being cultured for two to three days at 39.5° C.

Additional Material

CBCs grown at temperatures higher than 37° C. proliferate more rapidly and are generally healthier than CBCs grown at 37° C. Cells grown at 39.5° C. divide 25-40% faster than cells grown at 37° C. These cells are still able to give rise to somatic composites and most likely germline composites.

To ensure proper development, eggs are transferred to an incubator set at 37.5 to 38.5° C. Coincidentally, the rate of cell division slows when the egg is laid.

Drugs and Concentrations Used

Several drugs: blasticidin, puromycin and G418 were tested on the BDC culture and several feeder cultures. The results show that the BDCs have high tolerance against drug blasticidin and G418, which means to kill BDCs need longer time and high concentration. Different concentrations of puromycin from 0.3 ug/ml, 0.5 ug/ml and 0.8 ug/ml were tested. At 0.3 ug/ml level, the most CBC colonies can live above five days but at 0.5 ug/ml level all CBC colonies die. It was used at 0.5 ug/ml concentration. We also found the STOs can survive at 0.5 ug/ml puromycin medium for above 8 days at 39.5° C. culture condition even though STOs do not harbor a puromycin resistance gene (pac). Therefore we discovered that STOs fortuitously resistant to puromycin.

Methods to Dissociate the CBCs Colonies

Several enzymes and non-enzyme solutions were tested to dissociate the CBC colonies for their subculture. These enzymes include trypsin, collagenase, dispase II. The results show that most of the CBCs dissociated by these enzymes were differentiated after subculture even using BDC-CEE medium. Non-enzyme solution from JRH BIOSCIENCES (Cat No 59226-77p) and cell dissociation solution non-enzymatic from SIGMA (Cat No C-1544) were used to dissociate the CBC colonies, but both did not work well to dissociate the CBC colonies. The PBS with EDTA and PBS with EGTA at different concentrations were tested to dissociate the CBC colonies and they did not work. Pipetting method is a mechanical method to dissociate the CBC colonies. It did not work as well as trypsin method but the cells and cell clumps can be sub-cultured in BDC-CEE medium with very less differentiation after dissociations of the CBC colonies. Example: Put CBC colonies in 1 volume culture medium; set pipette man at 0.7 volume; then pipette up and down for 30 to 50 times. Transfer suspension into a culture well.

Use of Chicken Embryo Extract in CBCs Culture (e.g., Continous CBCs Culture).

Many nutrition factors were tested to find the best medium to continuously culture CBCs without differentiation. Surprisingly, chicken embryo extract as an additive of the medium has the ability to keep most CBC colonies un-differentiated for number of passages, for example, up to seven passages or more. This discovery provides for culturing a single CBC colony to a sufficient amount of cells for production of composite chickens.

Some factors that assist in culturing and subculturing BDCs and CBCs without differentiation for gene targeting or random insertion appear to include: STOs as a feeder, CEE as an additive of the medium, culture at 39.5° C., puromycin as a selection drug and 0.5 ug per milliliter as its concentration and pipetting as a method to dissociate the CBC colonies for their subculture.

While this invention has been described with respect to various specific examples and embodiments, it is to be understood that the invention is not limited thereto and that it can be variously practiced with the scope of the following claims.

What is claimed is:

1. A method of producing an integrated transgene in an avian cell comprising:
introducing a nucleic acid comprising a non-lethal marker gene into an avian cell by electroporating; and
allowing the cell to undergo a cellular division; thereby producing an integrated transgene in an avian cell.

2. The method of claim 1 comprising allowing the cell to undergo a division in the presence of chick embryo extract.

3. The method of claim 1 wherein the transgene is stably integrated.

4. The method of claim 1 wherein the marker gene is a fluorescent expression marker.

5. The method of claim 1 wherein the marker is a fluorescent protein expression marker.

6. The method of claim 1 wherein the marker is an green fluorescent protein expression marker.

7. The method of claim 1 wherein the marker is an antibiotic resistance gene.

8. The method of claim 1 wherein the marker is puromycin resistance.

9. The method of claim 1 wherein the avian cell is a blastodermal cell.

10. The method of claim 1 wherein the electroporating introduces a double stranded break in a nucleic acid.

11. A method of producing a transgenic avian comprising injecting a cell of claim 1 into an avian embryo.

12. The method of claim 11 wherein the cell is injected into the embryo after passage.

13. The method of claim 11 wherein the embryo is a stage X embryo.

14. The method of claim 11 wherein a coding sequence of the transgene is expressed in the blood of the transgenic avian.

15. The method of claim 11 wherein a coding sequence of the transgene is expressed in the sperm of the transgenic avian.

16. The method of claim 11 wherein a polypeptide encoded by a coding sequence of the transgene is present in egg white produce by the transgenic avian.

17. The method of claim 11 wherein the coding sequence is for a light chain or a heavy chain of an antibody.

18. The method of claim 17 wherein the antibody is a human antibody.

19. The method of claim 11 wherein the coding sequence is for a cytokine.

20. The method of claim 19 wherein the cytokine is interferon.

21. A method of screening for nucleic acid integration in a cellular genome comprising:
transforming a nucleic acid comprising a marker into a recipient avian cell and
determining if the nucleic is present in an equal copy number in cells of a colony produced by the recipient avian cell.
thereby screening for nucleic acid integration in a cellular genome.

22. The method of claim 21 wherein the transforming is accomplished by electroporation.

23. The method 21 wherein the nucleic acid is DNA.

24. The method of claim 21 wherein an expression construct comprises the nucleic acid.

25. The method of claim 21 wherein the cell is an avian blastoderm cell.

26. The method of claim 21 wherein the marker is a fluorescent marker.

27. The method of claim 21 wherein the marker is a fluorescent protein expression marker.

28. The method of claim 21 wherein the marker is an green fluorescent protein expression marker.

29. The method of claim 21 wherein the determining if the nucleic is present in an equal copy number in cells of a colony produced by the recipient avian cell is accomplished based on light emission.

30. The method of claim 21 wherein the determining if the nucleic acid is present in an equal copy number in cells of a colony produced by the recipient avian cell is accomplished by determining if a marker is homogeneously present in cells of a colony produced by the recipient cell.

31. The method of claim 29 wherein the marker is present homogeneously in cells of a colony produced by the recipient cell indicating the nucleic acid is integrated in the genome of the recipient host cell.

32. The method of claim 29 wherein the marker is present non-homogeneously in cells of a colony produced by the recipient cell indicating the nucleic acid is not integrated in the genome of the recipient host cell.
